SEQUENCE LISTING (13 sequences)

SEQ ID NO: 1 - DNA, 20 bp, synthetic construct
  source 1..20: mol_type = other DNA
tccatgacgttcctgacgtt

SEQ ID NO: 2 - DNA, 20 bp, synthetic construct
  source 1..20: mol_type = other DNA
ggggtcaacgttgagggggg

SEQ ID NO: 3 - DNA, 20 bp, synthetic construct
  source 1..20: mol_type = other DNA
gggggacgatcgtcgggggg

SEQ ID NO: 4 - DNA, 21 bp, synthetic construct
  source 1..21: mol_type = other DNA
ggggacgacgtcgtggggggg

SEQ ID NO: 5 - DNA, 22 bp, synthetic construct
  source 1..22: mol_type = other DNA
tgactgtgaacgttcgagatga

SEQ ID NO: 6 - DNA, 20 bp, synthetic construct
  source 1..20: mol_type = other DNA
tccatgacgttcctgatgct

SEQ ID NO: 7 - DNA, 24 bp, synthetic construct
  source 1..24: mol_type = other DNA
tcgtcgttttgtcgttttgtcgtt

SEQ ID NO: 8 - DNA, 19 bp, synthetic construct
  source 1..19: mol_type = other DNA
tcgttgtcgttttgtcgtt

SEQ ID NO: 9 - DNA, 26 bp, synthetic construct
  source 1..26: mol_type = other DNA
tcgcgacgttcgcccgacgttcggta

SEQ ID NO: 10 - DNA, 22 bp, synthetic construct
  source 1..22: mol_type = other DNA
tcgtcgttttcggcgcgcgccg

SEQ ID NO: 11 - DNA, 25 bp, synthetic construct
  source 1..25: mol_type = other DNA
tcgtcgtcgttcgaacgacgttgat

SEQ ID NO: 12 - DNA, 29 bp, synthetic construct
  source 1..29: mol_type = other DNA
tcgcgaacgttcgccgcgttcgaacgcgg

SEQ ID NO: 13 - protein, 8 aa, synthetic construct
SIINFEKL

(19) United States
(12) Patent Application Publication
(10) Pub. No.: US 2026/0284184 A1
(43) Pub. Date: Sep. 24, 2026

(54) BIODEGRADABLE SCAFFOLDS FOR ENHANCING VACCINE EFFICACY

(71) Applicant: The Regents of the University of California, Oakland, CA (US)

(72) Inventors: Nisarg Shah, San Diego, CA (US); Matthew Kerr, Carlsbad, CA (US)

(21) Appl. No.: 19/474,126

(22) PCT Filed: Apr. 10, 2024
(86) PCT No.: PCT/US2024/023874

§ 371 Date: Oct. 9, 2025

Related U.S. Application Data
(60) Provisional application No. 63/458,616, filed on Apr. 11, 2023.

(51) Int. Cl. A61K 39/385; A61K 39/00; A61K 39/39; A61K 47/36; A61P 37/04

(52) U.S. Cl. CPC A61K 39/385; A61K 39/0011; A61K 39/39; A61K 47/36; A61P 37/04; A61K 2039/545; A61K 2039/55522; A61K 2039/645

(57) ABSTRACT

Scaffolds and materials which provide for sustained release of vaccine components upon delivery to a subject, thus resulting enhanced immune response compared to bolus vaccination strategies.

### CROSS REFERENCE

This application claims the benefit of U.S. Provisional Application No. 63/458,616 filed Apr. 11, 2023, which application is incorporated herein by reference in its entirety

### GOVERNMENT SPONSORSHIP

This invention was made with government support under CA153915, and HL 164055 awarded by the National Institutes of Health, and ECCS2025752 awarded by the National Science Foundation. The government has certain rights in the invention.

### SEQUENCE LISTING

The instant application contains a Sequence Listing which has been submitted electronically in ASCII format and is hereby incorporated by reference in its entirety. Said ASCII copy, created on Apr. 10, 2024 is named 24978-0913_SL.xml and is 12,292 bytes in size.

### BACKGROUND

Biomaterial-based vaccines have been shown to effectively address many challenges with conventional bolus vaccination strategies. In particular, immunization strategies focused on sustained release of vaccine components antigen and adjuvant have been demonstrated to induce a more potent, durable protective immune response compared to bolus vaccination¹⁻⁷. These studies have strongly supported a key role of sustained release of vaccine components in enhancing the immune response. While effective at inducing an immune response, the formulation itself could include components that could result in a persistent foreign body response (FBR), such as those that use long lasting polymers^{8,9}, that could be limiting in clinical settings^{8,10}. On the other hand, a degradable biomaterial-based vaccine which maximizes activation of the adaptive immune responses while avoiding a long-lasting FBR could be a potential alternative. There thus exists a need for materials which can provided sustained release of vaccine components and overcome these limitations.

### SUMMARY OF THE INVENTION

In one aspect, the instant disclosure relates to materials which provide for sustained release of vaccine components in order to better mimic antigen and adjuvant exposure duration of natural infections, which may be associated with enhanced vaccine efficacy compared to traditional bolus vaccination strategies.

In an embodiment, the instant disclosure provides a biodegradable hyaluronic acid (HA)-scaffold, referred to herein as HA cryogel, which mediates sustained antigen and adjuvant release in vivo leading to a durably protective adaptive immune response. A formulation which enhances the immune response while minimizing the inflammation associated with the foreign body response is provided, referred to as CpG-OVA-HAC2 herein. Dose escalation studies with CpG-OVA-HAC2 demonstrate that both the antibody and T cell responses are dose-dependent. In some instances, the responses are strongly dependent on competency of neutrophils to perform oxidative burst. In immunodeficient posthematopoietic stem cell transplant mice, immunization with CpG-OVA-HAC2 elicited a more rapid antibody response, three orders of magnitude higher than dose-matched bolus vaccine. In a melanoma mouse model, CpG-OVA-HAC2 induced dose-responsive prophylactic protection, slowing the tumor growth rate and enhancing overall survival. Upon re-challenge, none of the mice developed new tumors suggesting the development of robust immunological memory and long-lasting protection against repeat infections. The results from this work show that the materials provided herein enhance immune responses to antigens.

Thus, one aspect described herein is a sustained release vaccine composition, comprising a biodegradable scaffold; an antigen, and an adjuvant, wherein the antigen and adjuvant are disposed within the biodegradable scaffold, and wherein degradation of the biodegradable scaffold results in release of the antigen and the adjuvant from the biodegradable scaffold. Also described herein are pharmaceutical compositions comprising the sustained release vaccine composition, in particular those formulated for subcutaneous administration. Further provided herein are methods of performing a vaccination with the compositions described herein which comprise administering the composition to a subject.

Further provided are methods of preparing a sustained release vaccine composition described herein, comprising cross-linking a plurality of biodegradable polymers in the presence of an antigen and an adjuvant to provide a biodegradable scaffold with the antigen and adjuvant disposed therein.

### BRIEF DESCRIPTION OF THE DRAWINGS

FIG. 1A shows a schematic depicting HA cryogel vaccine formulation.

FIG. 1B shows confocal microscopy images, overhead and side views, depicting hydrated Cy5-conjugated HACJ (HAC1:Cy5) and HAC2:Cy5 incubated with 10 micron FITC-labeled microparticles. Scale bar=100 micron.

FIG. 1C shows scanning electron microscope (SEM) images of HA cryogels, HAC1 and HAC2. Left scale bar=2 mm, right scale bar=300 micron.

FIG. 1D shows in vitro degradation kinetics of OVA-encapsulated Cy5-labeled HAC1 (OVA-HAC1:Cy5) and HAC2 (OVA-HAC1:Cy5) in hyaluronidase 2 (HYAL2) solution. Data represents mean±s.d. of n=4 cryogels. Data compared using two-way ANOVA with Bonferroni's multiple comparison test.

FIG. 1E shows representative in vivo imaging system (IVIS) fluorescence images of OVA-HAC1:Cy5 and OVA-HAC2:Cy5 degradation.

FIG. 1F shows measurements of OVA-HAC1:Cy5 and OVA-HAC2:Cy5 degradation in vivo by quantification of total radiant efficiency normalized to initial day 3 time point. Data represents mean±s.e.m. of n=5 mice. Data compared using two-way ANOVA with Bonferroni's multiple comparison test.

FIG. 1G shows quantification of in vitro OVA release from OVA encapsulated HAC1 (OVA-HAC1) and OVA-HAC2 in either phosphate buffered saline or HYAL2 solution. Comparison of PBS and HYAL2 release was conducted by pooling measurements for OVA-HAC1 and OVA-HAC2. Data represents mean±s.d. of n=4 cryogels. Data compared using two-way ANOVA with Bonferroni's multiple comparison test.

FIG. 1H shows representative IVIS fluorescence images of Cy5-conjugated OVA (OVA:Cy5) encapsulated HAC1 (OVA:Cy5-HAC1) and OVA:Cy5-HAC2.

FIG. 1I shows measurements of OVA:Cy5 release from HAC1 and HAC2 by quantification of total radiant efficiency normalized to initial 6-hour time point. Data in i represents mean±s.e.m. of n=4 mice. Data compared using two-way ANOVA with Bonferroni's multiple comparison test.

FIG. 2A shows a workflow schematic for assessing innate immune cell infiltration in OVA-HAC.

FIG. 2B shows representative flow cytometry plots depicting gating strategy to determine cellular identity of CD45+CD11b+Ly6G+ (neutrophil), CD45+CD11b+Ly6G-CD115+ (monocyte), CD45+CD11b+Ly6G-CD115-F4/80+ (macrophage), and CD45+CD11b+Ly6G-CD115−F4/80−CD11c+ dendritic cells (DCs).

FIG. 2C shows quantification of total CD45+CD11b+ (myeloid) cells. Data represents mean±s.d. of n=9 cryogels.

FIG. 2D shows infiltrating immune cells plotted as a percentage of myeloid cells. Data represents mean±s.d. of n=9 cryogels.

FIGS. 2E-H show quantification of total numbers of neutrophils (FIG. 2E), monocytes (FIG. 2F), macrophages (FIG. 2G), and DCs (FIG. 2H). Data represents mean s.d. of n=9 cryogels.

FIG. 2I shows hematoxylin and eosin (H&E) stained histological sections of explanted OVAHAC1 and OVA-HAC2 7-days post-injection. Full view scale bar=800 micron, magnified scale bar=100 micron.

FIG. 2J shows quantification of cellular density in the sections from H&E slides. Data represents mean±s.d. of n=4 cryogels.

FIG. 2K shows quantification of fibrotic capsule thickness in the sections from H&E slides. Data represents mean±s.d. of n=12 measurements (4 measurements per cryogel).

FIG. 2L shows an assessment of anti-OVA IgG1 antibody titers in serum of mice which received a single injection of OVA-HAC1 or OVA-HAC2, administered in a prime and boost setting 11 days apart. Data represents mean±s.d. of n=5 mice. Data in c, e-h, j, and k compared using student's t-test. Data compared using two-way ANOVA with Bonferroni's multiple comparison test.

FIG. 3A shows quantification of in vitro CpG release from CpG and OVA encapsulated HA cryogel from supplier 2 (CpG-OVA-HAC2) in both 1×PBS and hyaluronidase 2 (HYAL2) solution. Data represents mean±s.d. of n=4 cryogels. Data compared using two-way ANOVA with Bonferroni's multiple comparison test.

FIG. 3B shows quantification of in vitro GM-CSF release from GM-CSF and OVA encapsulated HAC2 (GMCSF-OVA-HAC2) in both 1×PBS and HYAL2 solution. Data represents mean±s.d. of n=4 cryogels. Data compared using two-way ANOVA with Bonferroni's multiple comparison test.

FIG. 4A shows an overview schematic depicting for in vivo degradation study.

FIGS. 4B-E show representative IVIS fluorescence images of cryogel degradation and quantification by measuring total radiant efficiency normalized to initial day 3 time point of OVA-HAC2 (FIG. 4B), GMCSF-OVA-HAC2 (FIG. 4C), CpG-OVA-HAC2 (FIG. 4D), and GM-CSF and CpG encapsulated OVA-HAC2 (GM-CSF-CpG-OVA-HAC2) (FIG. 4E). Data in FIGS. 4E-4H represents mean±s.e.m. of n=5 mice. Data in FIGS. 4E-4H compared two-way ANOVA with Bonferroni's multiple comparison test on prime vaccine degradation curves.

FIG. 5A shows a workflow schematic for assessing innate immune cell infiltration in OVA-HAC2, GMCSF and OVA encapsulated HAC2 (GMCSF-OVA-HAC2), CpG and OVA encapsulated HAC2 (CpG-OVA-HAC2), and GM-CSF, CpG, and OVA encapsulated HAC2 (GMCSF-CpG-OVA-HAC2).

FIG. 5B shows quantification of total CD45+CD11b+ (myeloid) cells in cryogels removed 10-days post-injection. Data sets provided from left to right are for HAC2, OVA-HAC2, GMCSF-OVA-HAC2, CpG-OVA-HAC2, and GMCSF-CpG-OVA-HAC2, respectively. Data represents mean±s.d. of n=5 cryogels. Data compared using one-way ANOVA with Dunnet's multiple comparison.

FIG. 5C show infiltrating immune cell lineages plotted as a percentage of myeloid cells in cryogels removed 10-days post-injection. Data represents mean±s.d. of n=5 cryogels.

FIGS. 5D-G show quantification of total numbers of CD45+CD11b+Ly6G+ (neutrophils) (FIG. 5D), CD45+CD11b+Ly6GCD115+ (monocytes) (FIG. 5E), CD45+CD11b+Ly6GCD115−F4/80+ (macrophages) (FIG. 5F), and CD45+CD11b⁺Ly6⁻GCD115−F4/80−CD11c+ (dendritic) cells (DCs) (FIG. 5G) in cryogels removed 10-days post-injection. Data represents mean±s.d. of n=5 cryogels. Data sets provided from left to right for each of FIGS. 5D-5G are for HAC2, OVA-HAC2, GMCSF-OVA-HAC2, CpG-OVA-HAC2, and GMCSF-CpG-OVA-HAC2, respectively. Data in FIGS. 5E-5G compared using one-way ANOVA with Dunnet's multiple comparison. Data in FIG. 5D was compared using Kruskal-Wallis test with Dunnet's multiple comparison.

FIG. 5H shows hematoxylin and eosin (H&E) stained histological sections of explanted OVA-HAC2, GMCSF-OVA-HAC2, CpG-OVA-HAC2, and GMCSF-CpG-OVA-HAC2 10-days post-injection. Full view scale bar=800 micron, magnified scale bar=100 micron.

FIG. 5I shows quantification of cellular density in the sections from H&E slides. Data represents mean±s.d. of n=8-10 cryogels. Data sets provided from left to right are for HAC2, OVA-HAC2, GMCSF-OVA-HAC2, CpG-OVA-HAC2, and GMCSF-CpG-OVA-HAC2, respectively. Data compared using one-way ANOVA with Dunnet's multiple comparison.

FIG. 5J shows quantification of fibrotic capsule thickness in the sections from H&E slides. Data sets provided from left to right are for HAC2, OVA-HAC2, GMCSF-OVA-HAC2, CpG-OVA-HAC2, and GMCSF-CpG-OVA-HAC2, respectively. Data represents mean s.d. of n=20 measurements (4 measurements per cryogel). Data compared using one-way ANOVA with Dunnet's multiple comparison.

FIG. 5K shows an assessment of anti-OVA IgG1 antibody titers in serum of mice which received OVAHAC2, GMCSF-OVA-HAC2, CpG-OVA-HAC2, or GMCSF-CpG-OVA-HAC2 administered in a prime and boost setting 11-days apart. Data represents mean±s.d. of n=5 mice. Data compared using two-way ANOVA with Bonferroni's multiple comparison test.

FIG. 6A shows representative in vivo imaging system (IVIS) fluorescence images of CyS-labeled CpG (CpG:Cy5) and OVA encapsulated within HA cryogels from supplier 2 (CpG:Cy5-OVA-HAC2) and measuring degradation by quantification of total radiant efficiency normalized to initial 6-hour time point. Data represents mean±s.e.m. of n=5 mice.

FIG. 6B shows an assessment of anti-OVA IgG1 antibody titers in serum of mice which received CpG-OVA-HAC2 administered as a single dose prime, two-dose prime, single dose prime and boost administered 11-days apart, or two-dose prime and boost administered 11-days apart. Data represents mean±s.d. of n=5 mice. Data compared pairwise using two-way ANOVA with Bonferroni's multiple comparison test.

FIG. 6C shows representative flow cytometry plots in axillary draining lymph nodes (LNs) to depicting gating strategy to assess CD45+B220− CD8+SIINFEKL+ cells (OVA-specific cytotoxic T cells).

FIG. 6D shows percentage of OVA-specific cytotoxic T cells of total CD45+B220-CD8+ cells (cytotoxic T cells) in axillary draining LNs. Data represents mean s.d. of n=5 mice. Data compared using one-way ANOVA with Dunnet's multiple comparison.

FIG. 6E shows representative IVIS fluorescence images of CpG and OVA encapsulated within Cy5-conjugated HAC2 (CpG-OVA-HAC2:Cy5) in B6 and gp91phox− mice.

FIG. 6F shows an assessment of anti-OVA IgG1 antibody titers in serum of B6 and gp91phox− mice which received 2 CpG-OVA-HAC2:Cy5 as a prime and boost. Data represents mean±s.d. of n=5 mice. Data compared pairwise using two-way ANOVA with Bonferroni's multiple comparison test.

FIG. 6G shows an overview schematic for assessing anti-OVA IgG1 antibody titers post autologous hematopoietic stem cell transplant (HSCT) mice.

FIG. 6H shows an anti-OVA IgG1 antibody titers in serum of mice following 2 prime and boost vaccination of either 2 bolus CpG+OVA vaccination or 2 CpG-OVA-HAC2 post-HSCT. Data represents mean±s.d. of n=7 mice. Data compared pairwise using two-way ANOVA with Bonferroni's multiple comparison test.

FIG. 7A shows an overview schematic for assessing prophylactic immunization in mediating protection against B16-OVA melanoma.

FIGS. 7B-7D show progression-free survival (FIG. 7B), tumor volume measured in individual mice (FIG. 7C), and overall survival (FIG. 7D). Mice were inoculated with 100K B16-OVA melanoma cells administered subcutaneously either in unvaccinated mice, or after two-dose bolus, single dose CpG-OVA-HAC2, and two-dose CpG-OVA-HAC2 administered as a prime and boost.

FIG. 7E shows quantification of anti-OVA IgG1 antibody titers in serum of vaccinated mice 6-weeks post prime and 3-weeks post tumor inoculation.

FIG. 7F shows an overview schematic for assessing therapeutic immunization in mediating protection against B16-OVA melanoma.

FIG. 7G shows tumor volume measured in individual mice in a therapeutic immunization experiment.

FIG. 7H shows overall survival of mice in a therapeutic immunization experiment.

### DETAILED DESCRIPTION

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the exemplary methods, devices, and materials are described herein.

### Definitions

As used herein, the terms “comprises,” “comprising,” “includes,” “including,” “has,” “having,” “contains”, “containing,” “characterized by,” or any other variation thereof, are intended to encompass a non-exclusive inclusion, subject to any limitation explicitly indicated otherwise, of the recited components. For example, a fusion protein, a pharmaceutical composition, and/or a method that “comprises” a list of elements (e.g., components, features, or steps) is not necessarily limited to only those elements (or components or steps), but may include other elements (or components or steps) not expressly listed or inherent to the fusion protein, pharmaceutical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a fusion protein, pharmaceutical composition, and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

It is understood that aspects and embodiments of the invention described herein include “consisting” and/or “consisting essentially of” aspects and embodiments.

It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Values or ranges may be also be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In embodiments, “about” can be used to mean, for example, within 10% of the recited value, within 5% of the recited value, or within 2% of the recited value.

The term “at least” prior to a number or series of numbers is understood to include the number adjacent to the term “at least”, and all subsequent numbers or integers that could logically be included, as clear from context. When at least (or similar such term, such as at most, etc.) is present before a series of numbers or a range, it is understood that “at least” can modify each of the numbers in the series or range.

As used herein, “patient” or “subject” means a human or animal subject to be treated.

As used herein the term “pharmaceutical composition” refers to pharmaceutically acceptable compositions, wherein the composition comprises a pharmaceutically active agent, and in some embodiments further comprises a pharmaceutically acceptable carrier. In some embodiments, the pharmaceutical composition may be a combination of pharmaceutically active agents and carriers.

The term “combination” refers to either a fixed combination in one dosage unit form, or a kit of parts for the combined administration where one or more active compounds and a combination partner (e.g., another drug as explained below, also referred to as “therapeutic agent” or “co-agent”) may be administered independently at the same time or separately within time intervals. In some circumstances, the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g., a compound and a combination partner, are both administered to a patient simultaneously in the form of a single entity or dosage. The term “non-fixed combination” means that the active ingredients, e.g., a compound and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

As used herein the term “pharmaceutically acceptable” means approved by a regulatory agency of the Federal or a state government or listed in the U.S. Pharmacopoeia, other generally recognized pharmacopoeia in addition to other formulations that are safe for use in animals, and more particularly in humans and/or non-human mammals.

As used herein the term “pharmaceutically acceptable carrier” refers to an excipient, diluent, preservative, solubilizer, emulsifier, adjuvant, and/or vehicle with which demethylation compound(s), is administered. Such carriers may be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may also be a carrier. Methods for producing compositions in combination with carriers are known to those of skill in the art. In some embodiments, the language “pharmaceutically acceptable carrier” is intended to include any and all solvents, dispersion media, coatings, isotonic and absorption delaying agents, and the like, compatible with pharmaceutical administration. The use of such media and agents for pharmaceutically active substances is well known in the art. See, e.g., Remington, The Science and Practice of Pharmacy, 20th ed., (Lippincott, Williams & Wilkins 2003). Except insofar as any conventional media or agent is incompatible with the active compound, such use in the compositions is contemplated.

As used herein, “therapeutically effective amount” refers to an amount of a pharmaceutically active compound(s) that is sufficient to treat or ameliorate, or in some manner reduce the symptoms associated with diseases and medical conditions. When used with reference to a method, the method is sufficiently effective to treat or ameliorate, or in some manner reduce the symptoms associated with diseases or conditions. For example, an effective amount in reference to diseases is that amount which is sufficient to block or prevent onset; or if disease pathology has begun, to palliate, ameliorate, stabilize, reverse or slow progression of the disease, or otherwise reduce pathological consequences of the disease. In any case, an effective amount may be given in single or divided doses.

As used herein, the terms “treat,” “treatment,” or “treating” embraces at least an amelioration of the symptoms associated with diseases in the patient, where amelioration is used in a broad sense to refer to at least a reduction in the magnitude of a parameter, e.g. a symptom associated with the disease or condition being treated. As such, “treatment” also includes situations where the disease, disorder, or pathological condition, or at least symptoms associated therewith, are completely inhibited (e.g. prevented from happening) or stopped (e.g. terminated) such that the patient no longer suffers from the condition, or at least the symptoms that characterize the condition.

As used herein, and unless otherwise specified, the terms “prevent,” “preventing” and “prevention” refer to the prevention of the onset, recurrence or spread of a disease or disorder, or of one or more symptoms thereof. In certain embodiments, the terms refer to the treatment with or administration of a compound or dosage form provided herein, with or without one or more other additional active agent(s), prior to the onset of symptoms, particularly to subjects at risk of disease or disorders provided herein. The terms encompass the inhibition or reduction of a symptom of the particular disease. In certain embodiments, subjects with familial history of a disease are potential candidates for preventive regimens. In certain embodiments, subjects who have a history of recurring symptoms are also potential candidates for prevention. In this regard, the term “prevention” may be interchangeably used with the term “prophylactic treatment.”

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with one or more other agent(s), which provides a prophylactic benefit in the prevention of the disease. The term “prophylactically effective amount” can encompass an amount that improves overall prophylaxis or enhances the prophylactic efficacy of another prophylactic agent.

As used herein, and unless otherwise specified, the term “subject” is defined herein to include animals such as mammals, including, but not limited to, primates (e.g., humans), cows, sheep, goats, horses, dogs, cats, rabbits, rats, mice, and the like. In specific embodiments, the subject is a human. The terms “subject” and “patient” are used interchangeably herein in reference, for example, to a mammalian subject, such as a human.

As used herein, the term “sustained release” refers to an agent-containing formulation, such as a composition or scaffold as described herein, in which complete release of the agent is not immediate, i.e., with a “sustained release” formulation, administration does not result in immediate release of the entirety of the agent. The term is used interchangeably with “nonimmediate release” as defined in Remington: The Science and Practice of Pharmacy, Nineteenth Ed. (Easton, PA: Mack Publishing Company, 1995). The sustained release may be a slower release of a portion of the relevant agent (e.g., an antigen and/or adjuvant as described herein) following an initial quick release of a portion of the agent (i.e., following a “burst” phase). As such, only a portion of the agent within the scaffold need be released gradually over an extended period of time.

“Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In preferred embodiments, the compositions are administered by injection, e.g., subcutaneous injection.

A “linker” refers to a chemical moiety that covalently or non-covalently attaches a compound or substituent group to another moiety. Linkers are typically at least bifunctional chemical moieties. Generally a linker has no specific biological activity other than to, e.g., join chemical species together or to preserve some minimum distance or other spatial relationship between such species. However, the constituents of a linker may be selected to influence some property of the linked chemical species such as three-dimensional conformation, net charge, hydrophobicity, etc. Exemplary linkers include, e.g., oligopeptides, oligopolyamides, oligoethyleneglycerols, oligoacrylamides, alkyl chains, or the like.

The term “attached” or “conjugated” refers to interactions and/or states in which material or compounds are connected or otherwise joined with one another. These interactions and/or states are typically produced by, e.g., covalent bonding, ionic bonding, chemisorption, physisorption, and combinations thereof. In preferred embodiments, a conjugated product will have two separate moieties linked by covalent binding.

### Sustained Release Vaccine Compositions

Provided herein in are sustained release vaccine compositions. Such sustained release vaccine compositions comprise a biodegradable scaffold into which an antigen and an adjuvant are deposited. Upon administration to the subject, the biodegradable scaffold breaks down, thereby providing sustained release of the antigen and adjuvant from the scaffold. This provides prolonged exposure to the antigen and adjuvant which can increase vaccine efficacy substantially compared to traditional bolus strategies.

In embodiments, the biodegradable scaffold is preferably selected such that it can itself also provide some immune stimulating effect. For example, hyaluronic acid (HA) is preferably employed as the biodegradable scaffold (most preferably as a cryogel), as it has been shown that degradation (such as by endogenous hyaluronidases) results in low molecular weight HA fragments shown to activate toll-like receptors (TLRs). Thus, provided herein in certain embodiments are vaccine compositions which comprise a biodegradable scaffold which itself is capable of acting to enhance the immune system in conjunction with an additional adjuvant (e.g., CpG) and the antigen to which an immune response is desired.

### Biodegradable Scaffolds

A sustained release vaccine composition according to the instant disclosure comprises a biodegradable scaffold. The biodegradable scaffold is made from a material which is biodegradable (i.e., able to be broken down within a biological system, such as within the body of a subject). The biodegradable scaffold is preferably able to contain the active agents of the vaccine (e.g., the antigen and adjuvant). In embodiments, the active agents are released over time after administration of the scaffold as the scaffold biodegrades within the subject. In embodiments, the active agents (i.e., the antigen and adjuvant as described herein) are disposed within the biodegradable scaffold. (e.g., within pores of the biodegradable scaffold). In embodiments, degradation of the biodegradable scaffold results in release of the active components (i.e., antigen and the adjuvant) from the biodegradable scaffold over time.

Preferably, the material which makes up the biodegradable scaffold is non-toxic and/or non-immunogenic. As used herein, the term “biocompatible material” refers to any material that does not induce a significant immune response or deleterious tissue reaction, e.g., toxic reaction or significant irritation, over time when implanted into or placed adjacent to the biological tissue of a subject.

Exemplary biomaterials suitable for use as scaffolds in the present invention include glycosaminoglycan, silk, fibrin, MATRIGEL®, poly-ethyleneglycol (PEG), polyhydroxy ethyl methacrylate, polyacrylamide, poly (N-vinyl pyrolidone), (PGA), poly lactic-co-glycolic acid (PLGA), poly e-carpolactone (PCL), polyethylene oxide, poly propylene fumarate (PPF), poly acrylic acid (PAA), polyhydroxybutyric acid, hydrolysed polyacrylonitrile, polymethacrylic acid, polyethylene amine, esters of alginic acid; pectinic acid; and alginate, fully or partially oxidized alginate, hyaluronic acid, carboxy methyl cellulose, heparin, heparin sulfate, chitosan, carboxymethyl chitosan, chitin, pullulan, gellan, xanthan, collagen, gelatin, carboxymethyl starch, carboxymethyl dextran, chondroitin sulfate, cationic guar, cationic starch, and combinations thereof. Preferably, a biodegradable scaffold of a sustained release vaccine composition as described herein comprises polymers of hyaluronic acid or a derivative thereof.

In embodiments, the biodegradable scaffold is made from hyaluronic acid. Hyaluronic acid (HA; conjugate base hyaluronate), is an anionic, nonsulfated glycosaminoglycan distributed widely throughout connective, epithelial, and neural tissues. One of the chief components of the extracellular matrix, hyaluronic acid contributes significantly to cell proliferation and migration. Natural hyaluronic acid is an important component of articular cartilage, muscular connective tissues, and skin. Hyaluronic acid is a polymer of disaccharides, composed of D-glucuronic acid and N-acetyl-D-glucosamine, linked via alternating β-(1→4) and β-(1→3) glycosidic bonds. Polymers of hyaluronic acid can range in size from 5,000 to 20,000,000 Da. Hyaluronic acid can also contain silicon.

Hyaluronic acid is energetically stable, in part because of the stereochemistry of its component disaccharides. Bulky groups on each sugar molecule are in sterically favored positions, whereas the smaller hydrogens assume the less-favorable axial positions. Hyaluronic acid can be degraded by a family of enzymes called hyaluronidases, which are present in many mammals, e.g., a human. Hyaluronic acid can also be degraded via non-enzymatic reactions. These include acidic and alkaline hydrolysis, ultrasonic disintegration, thermal decomposition, and degradation by oxidants. Due to its high biocompatibility and its common presence in the extracellular matrix of tissues, hyaluronic acid is used to form hydrogels, e.g., cryogels, as a biomaterial scaffold in tissue engineering research. Hyaluronic acid hydrogels are formed through cross-linking. Hyaluronic acid can form a hydrogel, e.g., cryogel, into a desired shape to deliver therapeutic molecules into a host. Hyaluronic acids, for use in the present compositions, can be cross-linked by attaching thiols, methacrylates, hexadecylamides, tyramines, and, (most preferably), click reagents. Hyaluronic acids can also be cross-linked directly with formaldehyde or with divinylsulfone. The term “hyaluronic acid,” includes unmodified hyaluronic acid or modified hyaluronic acid. Modified hyaluronic acid includes, but is not limited to, oxidized hyaluronic acid and/or reduced hyaluronic acid. The term “hyaluronic acid” or “hyaluronic acid polymers” may also include hyaluronic acid, e.g., unmodified hyaluronic acid, oxidized hyaluronic acid or reduced hyaluronic acid, or methacrylated hyaluronic acid or acrylated hyaluronic acid. Hyaluronic acid may also refer to any number of derivatives of hyaluronic acid. In embodiments, the hyaluronic acid polymers are unmodified except for reagents which enable the cross-linking of the polymers.

In embodiments, the biodegradable scaffold is prepared from hyaluronic acid polymers. In embodiments, the hyaluronic acid polymers are cross-linked together to form the biodegradable scaffold. In embodiments, the hyaluronic acid polymers have a molecular weight of about 0.1 MDa to about 10 MDa. In embodiments, the hyaluronic acid polymers have a molecular weight of from about 0.1 MDa to about 10 MDa, about 0.1 MDa to about 5 MDa, about 0.1 MDa to about 4 MDa, about 0.1 MDa to about 3 MDa, about 0.1 MDa to about 2 MDa, 0.5 MDa to about 10 MDa, about 0.5 MDa to about 5 MDa, about 0.5 MDa to about 4 MDa, about 0.5 MDa to about 3 MDa, about 0.5 MDa to about 2 MDa, 1 MDa to about 10 MDa, about 1 MDa to about 5 MDa, about 1 MDa to about 4 MDa, about 1 MDa to about 3 MDa, or about 1 MDa to about 2 MDa. In embodiments, the hyaluronic acid polymers have a molecular weight of about 0.5, 1, 1.5, 2, or 2.5 MDa. In embodiments, the molecular weight refers to a weight average molecular weight.

In embodiments, the scaffolds of present disclosure comprise one or more hydrogels (e.g., a hydrogel of hyaluronic acid). A hydrogel is a polymer gel comprising a network of cross-linked polymer chains. A hydrogel is usually a composition comprising polymer chains that are hydrophilic. The network structure of hydrogels allows them to absorb significant amounts of water. Some hydrogels are highly stretchable and elastic; others are viscoelastic. Hydrogel are sometimes found as a colloidal gel in which water is the dispersion medium. In certain embodiments, hydrogels are highly absorbent (they can contain over 99% water (v/v)) natural or synthetic polymers that possess a degree of flexibility very similar to natural tissue, due to their significant water content. In certain embodiments, a hydrogel may have a property that, when an appropriate shear stress is applied, the deformable hydrogel is dramatically and reversibly compressed (up to 95% of its volume), resulting in injectable macroporous preformed scaffolds. Preferred hydrogels of the instant disclosure are specifically cryogels as described herein, and are most preferably made from hyaluronic acid polymers.

Biodegradable scaffolds according to the instant disclosure can be prepared by cross-linking polymeric materials to form the scaffold. Many such cross-linking reagents are known in the art. In embodiments, biodegradable scaffolds herein are made by cross-linking polymeric materials using click chemistry techniques. In embodiments, the scaffold comprises click-hydrogels and/or click-cryogels. A click hydrogel or cryogel is a gel in which cross-linking between hydrogel or cryogel polymers is facilitated by click reactions between the polymers. Each polymer may contain one of more functional groups useful in a click reaction. Given the high level of specificity of the functional group pairs in a click reaction, active compounds can be added to the preformed device prior to or contemporaneously with formation of the hydrogel device by click chemistry. Non-limiting examples of click reactions that may be used to form click-hydrogels include Copper I catalyzed azide-alkyne cycloaddition, strain-promoted assize-alkyne cycloaddition, thiol-ene photocoupling, Diels-Alder reactions, inverse electron demand Diels-Alder reactions, tetrazole-alkene photo-click reactions, oxime reactions, thiol-Michael addition, and aldehyde-hydrazide coupling. Non-limiting aspects of click hydrogels are described in Jiang et al., 2014, Biomaterials, 35:4969-4985, the entire content of which is incorporated herein by reference.

In preferred embodiments, a biodegradable scaffold herein is comprised of a cryogel, most preferably a hyaluronic acid polymer cryogel. Cryogels are a class of materials with a highly porous interconnected structure that are produced using a cryotropic gelation (or cryogelation) technique. Cryogels also have a highly porous structure. Typically, active compounds are added to the cryogel device after the freeze formation of the pore/wall structure of the cryogel. Cryogels are characterized by high porosity, e.g., at least about 50, 55, 60, 65, 70, 75, 80, 85, 90, or 95% pores with thin pore walls that are characterized by high density of polymer cross-linking. As used herein, the term “porosity” refers to the percentage of the volume of pores to the volume of the scaffold. It is intended that values and ranges intermediate to the recited values are part of this invention. The walls of cryogels are typically dense and highly cross-linked, enabling them to be compressed through a needle into a subject without permanent deformation or substantial structural damage. In various embodiments, the pore walls comprise at least about 10, 15, 20, 25, 30, 35, or 40% (w/v) polymer. In other embodiments, the pore walls comprise about 10-40% polymer. In some embodiments, a polymer concentration of about 0.5-4% (w/v) (before the cryogelation) is used, and the concentration increases substantially upon completion of cryogelation. Non-limiting aspects of cryogel gelation and the increase of polymer concentration after cryogelation are discussed in Beduer et al., 2015 Advanced Healthcare Materials 4.2: 301-312, the entire content of which is incorporated herein by reference. In certain embodiments, cryogelation comprises a technique in which polymerization-cross-linking reactions are conducted in quasi-frozen reaction solution. Non-limiting examples of cryogelation techniques are described in U.S. Patent Application Publication No. 20140227327, published Aug. 14, 2014, the entire content of which is incorporated herein by reference. An advantage of cryogels compared to conventional macroporous hydrogels obtained by phase separation is their high reversible deformability. Cryogels may be extremely soft but can be deformed and reform their shape. In certain embodiments, cryogels can be very tough, can withstand high levels of deformations, such as elongation and torsion and can also be squeezed under mechanical force to drain out their solvent content. Exemplary hyaluronic acid cryogels compatible with the instant disclosure include those described in PCT Pub. No. WO 2022/099093, the entire content of which is incorporated herein by reference.

The shape of a biodegradable scaffold as described herein may be dictated by a mold and can thus take on any shape desired by the fabricator, e.g., various sizes and shapes (disc, cylinders, squares, strings, etc.) are prepared by cryogenic polymerization. Injectable cryogels can be prepared in the micrometer-scale to centimeter-scale. Exemplary volumes vary from a few hundred μm³ (e.g., about 100-500 μm³) to about 10 cm³. In certain embodiment, an exemplary scaffold composition is between about 100 μm³ to 100 mm³ in size. In various embodiments, the scaffold is between about 10 mm³ to about 100 mm³ in size. In certain embodiments, the scaffold is about 30 mm³ in size.

A biodegradable scaffold described herein is preferably injectable through a hollow needle. For example, the biodegradable scaffold is preferably able to pass through a needle (e.g., a 16 gauge (G) needle, e.g., having a 1.65 mm inner diameter). Other exemplary needle sizes are 16-gauge, an 18-gauge, a 20-gauge, a 22-gauge, a 24-gauge, a 26-gauge, a 28-gauge, a 30-gauge, a 32-gauge, or a 34-gauge needle. Injectable biodegradable scaffolds have been designed to pass through a hollow structure, e.g., very fine needles, such as 18-30 G needles. In certain embodiments, the biodegradable scaffold deforms during injection and returns to its approximately original geometry after traveling through a needle in a short period of time, such as less than about 10 seconds, less than about 5 seconds, less than about 2 seconds, or less than about 1 second. The scaffolds may be injected to a subject using any suitable injection device. For example, the scaffolds may preferably be injected using syringe through a needle. A syringe may include a plunger, a needle, and a reservoir that comprises compositions of the present disclosure.

In embodiments, the biodegradable scaffold is preferably porous. The pores are desirably of a size sufficient to allow cell infiltration (e.g., infiltration of the scaffold by one or more types of immune cells) after delivery to a subject. In embodiments, the biodegradable scaffold comprises pores having an average size of from about 1 micron to about 1000 micron (e.g., having an average pore size of 1 to 100 micron, 1 to 200 micron, 1 to 500 micron, 10 to 100 micron, 10 to 200 micron, 10 to 500 micron, 20 to 100 micron, 20 to 200 micron, 20 to 500 micron, 30 to 100 micron, 30 to 200 micron, or 30 to 500 micron). In embodiments, the biodegradable scaffold comprises pores having an average pore size of about 20 to 200 micron. In embodiments, the biodegradable scaffold comprises pores having an average pore size of about 20 to 100 micron. In embodiments, the biodegradable scaffold comprises pores having an average pore size of about 30 to 70 micron. In embodiments, the biodegradable scaffold comprise pores having an average pores size of about 20, 30, 40, 50, 60, 70, 80, 90, or 100 micron.

In embodiments, the pores are interconnected. Interconnectivity can be important to the function of the scaffold (e.g., a hydrogel or, more specifically, a cryogel scaffold as described herein), as without interconnectivity, water would become trapped within the gel. Interconnectivity of the pores permits passage of water (and other compositions such as cells and compounds) in and out of the structure. In embodiments, in a fully hydrated state, the scaffold (e.g., cryogel) comprises at least about 90% water (volume of water/volume of the scaffold) (e.g., between about 90-99%, at least about 92%, 95%, 97%, 99%, or more).

The biodegradable material may be degraded by physical or chemical action, e.g., level of hydration, heat, oxidation, or ion exchange or by cellular action, e.g., elaboration of enzyme, peptides, or other compounds by nearby or resident cells. In embodiments, the polymer scaffold comprises a biodegradable material that is a substrate for an endogenous enzyme. In embodiments, the polymer scaffold comprises a biodegradable material that is a substrate for an endogenous enzyme that catalyzes the degradation of a hyaluronic acid (HA). In embodiments, the polymer scaffold comprises a biodegradable material that is a substrate for a hyaluronoglucosidase, such as a hyaluronidase. In embodiments, the polymer scaffold comprises a biodegradable material that is a substrate for a hyaluronidase.

In embodiments, the scaffold can degrade at a predetermined rate based on a physical parameter selected from the group consisting of temperature, pH, hydration status, porosity, the cross-link density, type, and chemistry or the susceptibility of main chain linkages to degradation. In embodiments, the scaffold composition can degrade at a rate based on a level of immune deficiency and/or a level of functional innate leukocytes, including a level of activated neutrophils, in a subject. For example, a scaffold composition comprising hyaluronic acid (HA), as described herein, can degrade in a matter of days, e.g., 1, 2, 3, 4, 5, 7, 8, 9, 10, 11, 12, 13, 14, or 15 or more days, or can degrade in a matter of weeks, e.g., 1, 2, 3, 4, 5, 6, 7, 8, 9, or 10 or more weeks.

In embodiments, the scaffolds of the present disclosure are organized in a variety of geometric shapes (e.g., discs, beads, pellets), niches, planar layers (e.g., thin sheets). For example, discs of about 0.1-200 millimeters in diameter, e.g., 5, 10, 20, 40, or 50 millimeters may be implanted subcutaneously. The disc may have a thickness of 0.1 to 10 millimeters, e.g., 1, 2, or 5 millimeters. The discs are readily compressed or lyophilized for administration to a patient. An exemplary disc for subcutaneous administration has the following dimensions: 8 millimeters in diameter and 1 millimeter in thickness.

In embodiments, the scaffold can comprise materials that are non-biodegradable. Exemplary non-biodegradable materials include, but are not limited to, metal, plastic polymer, or silk polymer. In preferred embodiments, the majority of the scaffold (e.g., at least 80%, 85%, 90%, 95%, 96%, 97%, 98%, or 99% of the scaffold) is comprised of the biodegradable materials as described herein (e.g., hyaluronic acid polymers).

### Antigens

The sustained release vaccine compositions according to the instant disclosure comprise at least one antigen. The antigen is included within the vaccine composition in order to elicit a favorable immune response (e.g., eliciting antibodies to the antigen) from a subject towards that antigen upon administration. The antigen included can be the native antigen (e.g., the native form of the antigen as it appears on the substance to be neutralized by the elicited antibodies) or can be a variant thereof which is also effective to raise antibodies against the native antigen. Favorably, vaccination with the composition results in a robust antibody response to the antigen.

The antigen can be any desired antigen, but is preferably a water soluble antigen for compatible manufacturing and formulation of the sustained release vaccine composition (e.g., disposition within the biodegradable scaffold). The antigen can be of a variety of types, including without limitation proteins, peptides (e.g., small peptides of up to ~50 amino acids), polysaccharides, lipids, and/or nucleic acids. In embodiments, the antigen is a protein. The antigen can be derived from a variety of sources, including without limitation pathogens (e.g., viral, bacterial, fungal, and/or parasitic pathogens), cancer cells (e.g., biomarkers which are only expressed and/or upregulated in cancer cells such as tumor cells), toxins (e.g., cholera toxoid, tetanus toxoid, diphtheria toxoid, etc.), or other desired source.

In embodiments, the antigen is a cancer associated antigen. In embodiments, the antigen is a tumor associated antigen. In embodiments, the cancer or tumor associated antigen is a protein upregulated, overexpressed, preferentially expressed, and/or only expressed in a cancer or tumor cell, or is a portion of such an antigen (e.g., a portion of the relevant protein, such as an extracellular domain or fragment thereof). In embodiments, the cancer or tumor associated antigen is selected from alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-1, 2, and 3, neo-PAP, myosin class I, OS-9, pml-RARα fusion protein, PTPRK, K-ras, N-ras, Triosephosphate isomeras, Bage-1, Gage 3,4,5,6,7, GnTV, Herv-K-mel, Lage-1, Mage-A1,2,3,4,6,10,12, Mage-C2, NA-88, NY-Eso-1/Lage-2, SP17, SSX-2, and TRP2-Int2, MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGS), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, α-fetoprotein, 13HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), human EGFR protein or its fragments, HTgp-175, M344, MA-50, MG7-Ag, MOV18, NB\70K, NY-CO-1, RCAS1, SDCCAG16, TA-90 (Mac-2 binding protein\cyclophilin C-associated protein), TAAL6, TAG72, TLP, TPS, WT1, MUC1, LMP2, EGFRvIII, Idiotype, GD2, Ras mutant, p53 mutant, Proteinase3 (PR1), Survivin, hTERT, Sarcoma translocation breakpoints, EphA2, EphA4, LMW-PTP, PAP, ML-IAP, AFP, ERG (TMPRSS2 ETS fusion gene), NA17, PAX3, ALK, Androgen receptor, Cyclin B1, Polysialic acid, MYCN, RhoC, TRP-2, GD3, Fucosyl GMI, Mesothelin, sLe (animal), CYP1B1, PLAC1, GM3, BORIS, Tn, GloboH, NY-BR-1, RGS5, SART3, STn, Carbonic anhydrase IX, PAX5, OY-TES1, Sperm protein 17, LCK, HMWMAA, AKAP-4, XAGE 1, B7H3, Legumain, Tie 2, Page4, VEGFR2, MAD-CT-1, FAP, PDGFR-alpha, PDGFR-β, MAD-CT-2, Fos-related antigen 1, ERBB2, Folate receptor 1 (FOLR1 or FBP), IDH1, IDO, LY6K, fins-related tyro-sine kinase 1 (FLT1, best known as VEGFR1), KDR, PADRE, TA-CIN (recombinant HPV16 L2E7E6), SOX2, aldehyde dehydrogenase, OVA, and any derivative or fragment thereof. In embodiments, the cancer or tumor associated antigen is OVA, Trp-2, or gp100.

In embodiments, the antigen is a pathogen associated antigen. In embodiments, the antigen is a portion of a virus, such as a virus selected from Cowpoxvirus, Vaccinia virus, Pseudocowpox virus, Human herpesvirus 1, Human herpesvirus 2, Cytomegalovirus, Human adenovirus A-F, Polyomavirus, Human papillomavirus, Parvovirus, Hepatitis A virus, Hepatitis B virus, Hepatitis C virus, Human immunodeficiency virus, Orthoreovirus, Rotavirus, Ebolavirus, parainfluenza virus, influenza virus (e.g., H5N1 influenza virus, influenza A virus, influenza B virus, influenza C virus), Measles virus, Mumps virus, Rubella virus, Pneumovirus, Human respiratory syncytial virus, Rabies virus, California encephalitis virus, Japanese encephalitis virus, Hantaan virus, Lymphocytic choriomeningitis virus, Coronavirus, Enterovirus, Rhinovirus, Poliovirus, Norovirus, Flavivirus, Dengue virus, West Nile virus, Yellow fever virus and varicella.

In embodiments, the antigen is a portion of a bacterium, such as a bacterium selected from Anthrax (Bacillus anthracis), Brucella, Bordetella pertussis, Candida, Chlamydia pneumoniae, Chlamydia psittaci, Cholera, Clostridium botulinum, Coccidioides immitis, Cryptococcus, Diphtheria, Escherichia coli, Enterotoxigenic Escherichia coli, Haemophilus influenzae, Helicobacter pylori, Legionella, Leptospira, Listeria, Meningococcus, Mycoplasma pneumoniae, Mycobacterium, Pertussis, Pneumonia, Salmonella, Shigella, Staphylococcus, Streptococcus pneumoniae and Yersinia enterocolitica.

In embodiments, the antigen is derived from a protozoan, such as a protozoan selected from the genus Plasmodium (e.g., Plasmodium falciparum, Plasmodium malariae, Plasmodium vivax, Plasmodium ovale or Plasmodium knowlesi) which causes malaria.

### Adjuvants

In embodiments, the sustained release vaccine compositions according to the instant disclosure comprise at least one adjuvant. The type of adjuvant used is preferably one which can be disposed within the biodegradable scaffold and released therefrom as the scaffold degrades. In embodiments, the adjuvant is a protein, nucleic acid, or small molecule.

In embodiments, the adjuvant is an immunostimulatory protein. In embodiments, the adjuvant is selected from CRM197, interleukin-12, interleukin-15, NF-KB subunit p65/Rel A, T-bet transcription factor, PPE44/pCI-OVA, Cholera Toxin Subunit A, C-terminal Hsp70, GM-CSF, MyD88, TRIF, IRF1, AIRF1, IRF3, IRF7, Flagellin, TBK1, HMGB1, DAI/ZBP1, or ehMHD5. In embodiments, the adjuvant is GM-CSF. GM-CSF (granulocyte-macrophage colony-stimulating factor, Uniprot ID: Q52GZ7) is a glycoprotein secreted by a variety of immune cells (e.g., macrophages, T cells, mast cells, NK cells, endothelial cells, and fibroblasts) and can act as a dendritic cell maturation factor.

In embodiments, the adjuvant is a TLR agonist. In embodiments, the TLR agonist is an agonist of TLR2, TLR3, TLR4, TLR5, TLR7/8 and/or TLR9. In embodiments, the TLR agonist is a TLR9 agonist. In embodiments, the adjuvant is a CpG oligonucleotide which comprises a CpG motif. In embodiments, the CpG motif is a D type CpG, a Class B CpG, or a Class C CpG. In embodiments, the CpG motif is a Class B CpG. In embodiments, the CpG oligonucleotide comprises a sequence having at least 80%, 85%, 90%, or 95% sequence identity to the sequence set forth in 5′-TCC ATG ACG TTC CTG ACG TT-3′ (SEQ ID NO: 1, also referred to herein as ODN 1826). In some embodiments, the CpG oligonucleotide comprises the sequence set forth in SEQ ID NO: 1. In embodiments, the CpG oligonucleotide comprises an oligonucleotide sequence having at least 80%, 85%, 90%, 95%, or 100% identity to a sequence selected from the table below (SEQ ID NOs: 1-12). Preferably, the sequence retains at least one or all of the “CG” motifs of the sequences listed below.

### CpG Sequences

| Sequence | SEQ ID NO: | CpG name | Type |
|---|---|---|---|
| 5′-TCC ATG ACG TTC CTG ACG | 1 | ODN 1826 | Class B |
| TT-3′ | | | |
| 5′-ggGGTCAACGTTGAgggggg -3′ | 2 | ODN 1585 | Class |
| | | | A |
| 5′-ggGGGACGATCGTCgggggg-3′ | 3 | ODN 2216 | Class |
| | | | A |
| 5′-gggGACGACGTCGTGgggggg -3′ | 4 | ODN 2336 | Class |
| | | | A |
| 5′-TGACTGTGAACGTTCGAGATGA-3′ | 5 | ODN 1018 | Class B |
| 5′-tccatgacgttcctgatgct-3′ | 6 | ODN 1668 | Class B |
| 5′-tcgtcgttttgtcgttttgtcgtt-3′ | 7 | ODN 2006 | Class B |
| 5′ -tcg ttg tcg ttt tgt cgt t -3′ | 8 | ODN 2007 | Class B |
| 5′-tcg cga cgt tcg ccc gac gtt cgg ta-3′ | 9 | ODN D-SL01 | Class B |
| 5′-tcgtcgttttcggcgc:gcgccg-3′ | 10 | ODN 2395 | Class C |
| 5′-tcgtcgtcgttc:gaacgacgttgat-3′ | 11 | ODN M362 | Class C |
| 5′-tcg cga acg ttc gcc geg ttc gaa cgc | 12 | ODN D-SL03 | Class C |
| gg-3′ | | | |

### Release Characteristics and Activity

In embodiments, a sustained release vaccine composition provided herein releases the antigen and adjuvant from the biodegradable scaffold after administration in vivo. Favorably, this sustained release provides continued delivery of the antigen and adjuvant over time (e.g., over a period of at least 3, 4, 5, 6, 7, or more days), thereby eliciting a stronger antibody response from the subject.

In embodiments, the antigen and adjuvant are released from the biodegradable scaffold as the biodegradable scaffold is degraded. In embodiments, the biodegradable scaffold degrades over a period of at least 3, 4, 5, 6, 7, 8, 9, 10, 11, 12, 13, 14, or 15 days (e.g., until complete degradation) in vivo. In embodiments, the biodegradable scaffold degrades over a period of at least 10, 15, 20, 25, or 30 days in vivo. In embodiments, the biodegradable scaffold degrades over a period of from 5 to 30 days, 10 to 30 days, 15 to 30 days, 5 to 25 days, 10 to 25 days, 15 to 25 days, 5 to 20 days, 10 to 20 days, or 15 to 20 days in vivo. In embodiments, the biodegradable scaffold degrades over a period of at least one week, two weeks, three weeks, four weeks, five weeks, or six weeks. In embodiments, the biodegradable scaffold degrades over a period of at least one week. In embodiments, the antigen and adjuvant are released for the entire time in which the biodegradable scaffold degrades.

In embodiments, the biodegradable scaffold residence time in vivo is best measured as a degradation half-life (e.g., the time to achieve a 50% reduction in fluorescence intensity of a fluorescently labeled biodegradable scaffold). In embodiments, the biodegradable scaffold has a degradation half-life in vivo of at least one week, at least two weeks, at least three weeks, at least four weeks, at least five weeks, or at least six weeks. In embodiments, the biodegradable scaffold has a degradation half-life of at least two weeks.

In embodiments, the antigen and adjuvant are released from the biodegradable scaffold in a non-linear fashion and/or at different rates after delivery to a subject. For example, a bolus of the antigen and adjuvant may be released rapidly in a “burst” phase in which there is an initial spike of antigen and adjuvant released followed by as slower sustained release phase where the antigen and adjuvant are released at a slower rate over time. In embodiments, the antigen and adjuvant are released in a burst phase and a sustained release phase. In embodiments, the burst phase results in at least at least 10%, 20%, 30%, 40%, or 50% of the antigen and/or the adjuvant being released within the first 24 hours after administration. In embodiments, the sustained release phase comprises the time period after the initial burst (e.g., the remaining time for the rest of the biodegradable scaffold to degrade, such as the remaining time after the initial 24 hour period). In embodiments, at least 50%, 60%, 70%, or 80% of the antigen and/or adjuvant is released from the biodegradable scaffold within a week of administration.

In embodiments, the biodegradable scaffold becomes infiltrated with one or more cell types following administration. In embodiments, the biodegradable scaffold becomes infiltrated with one or more immune cell types following administration. The ratio of various immune cell types which infiltrate the biodegradable scaffold can vary depending on the adjuvant used. In embodiments, use of an adjuvant preferably reduces the number or ratio of monocytes which infiltrate the biodegradable scaffold as compared to a non-adjuvanted composition. In embodiments, the one or more immune cells comprise a lower proportion of monocytes compared to a corresponding composition without the adjuvant. In embodiments, it can be preferably to increase the number or ratio of neutrophils which infiltrate the scaffold following the administration.

In embodiments, the administration of the sustained release vaccine composition described herein does not elicit a strong foreign body response (FBR) in the subject. In embodiments, administration of the composition does not elicit a FBR in the subject. In embodiments, administration of the composition minimally induces one or more symptoms associated with the FBR (e.g., inflammation, formation of a foreign body granuloma, etc.). In embodiments, the administration of the composition does not elicit inflammation associated with FBR. Such effects can be modulated by the proper selection of adjuvant (e.g., a CpG oligonucleotide as described herein).

### Methods of Administration

In an aspect provided herein are methods of performing a vaccination in a subject comprising administering to the subject a sustained release vaccine composition as described herein. In embodiments, the vaccination is effective to mount a robust humoral response in the subject. In embodiments, the vaccination is effective to prevent infection or reinfection by a pathogen associated with the antigen (for a pathogen associated antigen) or prevent spread, prevent formation, or prevent redevelopment of a cancer (for a cancer associated antigen).

In embodiments, a composition as described herein is administered multiple times in order to best induce the desired immune response. For example, the vaccine may be administered in both prime and one or more boost doses. In embodiments, administration as described herein comprises a prime dose and at least one boost dose. In embodiments, the prime and boost dose are administered at least one week apart (e.g., 7, 8, 9, 10, 11, 12, 13, or 14 days apart). In embodiments, use of a prime and boost dosing strategy elicits a stronger humoral response than a single dose.

In embodiments, the vaccination can be performed as a single dose. In such cases, it may be desirable to use a higher dose than would be used in a prime and boost setting. In embodiments, it has been observed herein that use of a higher initial dose can elicit a comparable response compared to a prime and boost regimen, in particular where the initial dose contains the same levels of total antigen and adjuvant delivered.

In embodiments, the vaccine composition is administered via an injection. Exemplary injection routes included, for example, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In preferred embodiments, the vaccine composition is administered subcutaneously. Favorably, the vaccine compositions comprising the biodegradable scaffolds herein are capable of being delivered with standard needle and syringe configurations (e.g., to not require complex implantation or specialized equipment). For example, the vaccine compositions can be delivered with any standard needle size, such as 16-gauge, an 18-gauge, a 20-gauge, a 22-gauge, a 24-gauge, a 26-gauge, a 28-gauge, a 30-gauge, a 32-gauge, or a 34-gauge needles.

In embodiments, administration of the vaccine is capable of eliciting the desired humoral response within a rapid timeframe (e.g., reaching a therapeutically relevant level within 7 days, 10 days, 14 days, etc.) of either the first administration (for a single dose or for a prime-boost strategy) or after a second administration (e.g., for a prime-boost strategy).

### Methods of Manufacturing

Also provided herein are methods of manufacturing a sustained release vaccine composition according to the instant disclosure. Briefly, in embodiments, the method comprises forming a biodegradable scaffold as described herein in the presence of the antigen and the adjuvant, thus disposing the antigen and adjuvant within the biodegradable scaffold. In embodiments, the forming the biodegradable scaffold comprises generating a cryogel, such as those described in, for example, PCT Pub. No. WO 2022/099093 A1, incorporated herein by reference.

In embodiments, the method of manufacturing a sustained release composition comprises cross-linking at a temperature below 0° C. a plurality of biodegradable polymers in the presence of an antigen and an adjuvant. In embodiments, the method provides a biodegradable scaffold with the antigen and adjuvant disposed therein. Preferably, the biodegradable scaffold prepared is a hydrogel, most preferably a cryogel. In preferred embodiments, the biodegradable polymers comprise hyaluronic acid polymers (e.g., any of those described herein supra).

Preferably, the cross-linking prefers in a medium which is an aqueous medium. In embodiments, the cross-linking reaction is performed in a quasi-frozen reaction solution, such as those described in, for example, U.S. Pat. App. Pub. No. 2014/0227327, which is incorporated herein by reference. Such a biodegradable scaffold formed includes microporous structures (e.g., pores as provided herein above supra) into which the antigen and adjuvant can become deposited. Preferably, the microporous structures (e.g., pores as described herein) are sufficiently sized to allow infiltration by cells upon administration (e.g., from about 20 micron to about 200 micron). In embodiments, the cross-linking is performed at a temperature of about −20° C.

The polymers can be cross-linked according to any desirable chemistry. In embodiments, the cross-linking results from a reaction of click reagents attached to the biodegradable polymers. To prepare such cross-linkable precursors, a biodegradable polymer (e.g., hyaluronic acid polymers) can be functionalized with a first click reagent in one batch and a complementary click reagent in a second batch. The two batches can subsequently be mixed in the presence of the antigen and adjuvant to form the biodegradable scaffold. Exemplary click reagents are well known in the art, and include norbornene and tetrazine reagents capable of reacting with the biodegradable polymer. The click reagents are preferably conjugated to the biodegradable polymer, optionally through any appropriate linker.

### Examples

Hyaluronic acid (HA) is a polysaccharide-based polymer that is abundant in tissues including skin, cartilage, and synovial fluid. It has been widely studied in biomedical applications including drug delivery as HA can be modified to form a matrix to encapsulate drugs, such as growth factors and chemotherapeutic agents, and release them in a sustained manner via controlled degradation¹¹. It has been previously demonstrated that the degradation of HA can be immune-responsive, mediated by oxidative burst of neutrophils¹¹, and is also processed by endogenous hyaluronidases^{12,13} (HYAL). The degradation generates low molecular weight HA fragments which have been shown to activate toll-like receptors (TLRs). The use of HA as an adjuvant in vaccine formulations has been previously reported^{4,6}.

Herein, it was hypothesized that a HA-based vaccine formulation would enhance immune cell activation and sustain release of encapsulated vaccine components to generate a durable protective immune response. A macroporous injectable HA hydrogel, termed HA cryogel, had previously been developed¹¹. It was demonstrated that HA cryogels mediated sustained release of protein therapeutics to enhance recovery of innate immune cells¹¹. Herein, the inventors have extended these previous findings to further develop HA cryogels as depots for sustained release of vaccine components. After evaluating cryogel formulations generated from commercially sourced HA by immunophenotypic and histological assessments, low endotoxin HA, termed HAC2, was selected for evaluation with encapsulated model antigen OVA, adjuvants CpG-ODN 1826 (CpG) and granulocyte macrophage colony stimulating factor (GM-CSF). Both CpG and GM-CSF comparably enhanced the response to the vaccine, but GM-CSF increased inflammation and the FBR at the injection site. CpG-loaded OVA-HAC2 (CpG-OVA-HAC2) for further analysis and conducted dose ranging studies with the CpG-OVA-HAC2 were conducted. In healthy mice, the intensity of the adaptive immune response was dose dependent on CpG-OVA-HAC2. However, in gp91phox− mice, which model neutrophil dysfunction, the induction of antibodies was significantly delayed. In post-hematopoietic stem cell transplant (HSCT) mice, CpG-OVAHAC2 enhanced antibody induction three orders of magnitude greater than dose-matched bolus vaccination. In a B16-OVA melanoma mouse model, CpG-OVA-HAC2 slowed the tumor growth rate and enhanced tumor-free overall survival in a dose-dependent manner.

### Synthesis and Characterization of HA Cryogels

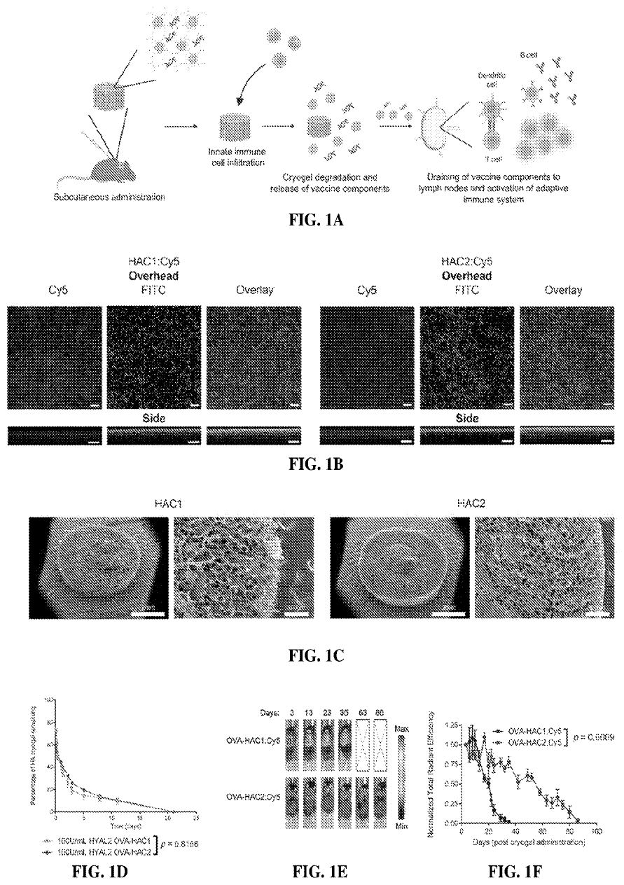

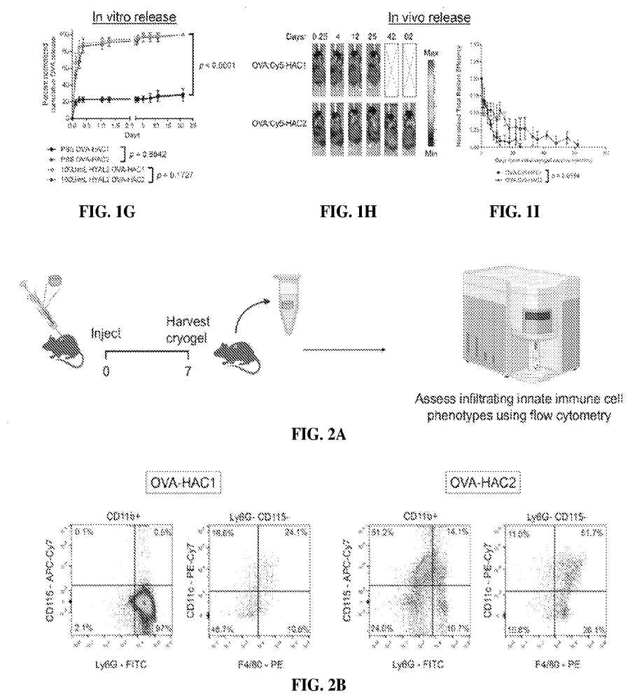

HA cryogels are formulated by first conjugating either tetrazine (Tz) amine or norbornene (Nb) methylamine to HA to form tetrazine-functionalized HA (HA-Tz) and norbornene functionalized HA (HA-Nb). Vaccine components were solubilized with the polymer solution prior to mixing and overnight incubation at −20° C. to form HA cryogel vaccines (FIG. 1A). HA was initially sourced from two commercial suppliers and resulting HA cryogels are referred to as HAC1 and HAC2 for HA sourced from supplier 1 and 2 respectively. For confocal imaging and in vivo degradation tracking, HA-Nb was reacted with Tz-Cy5 to form Cy5-labeled HA-Nb (Cy5-HA-Nb) which was mixed with HA-Tz to form Cy5-labeled HA cryogels, referred to as HAC1:Cy5 and HAC2:Cy5. To visualize pores and assess pore interconnectedness, HAC1:Cy5 and HAC2:Cy5 were incubated with fluorescein isothiocyanate (FITC)-labeled 10 micron diameter melamine resin particles and imaged using confocal microscopy. FITC-particles were co-localized with Cy5-labeled HA polymer to the confocal depth limit for HAC1:Cy5 and HAC2:Cy5 (FIG. 1B). Next, we lyophilized HA cryogels and measured surface porosity using scanning electron microscopy (SEM) (FIG. 1C). Average pore diameter for HAC1 and HAC2 were found to be 59.5±19.4 and 53.8±15.8 micron, respectively.

To test the magnitude of an adaptive immune response to the HA cryogel alone, 100 microgram of OVA was encapsulated in HAC1 and HAC2 (OVA-HAC1 and OVA-HAC2) and in vitro degradation kinetics of OVA-HAC1 and OVA-HAC2 were assessed by incubating cryogels in hyaluronidase 2 (HYAL2) solution. The degradation profiles of OVA-HAC1 and OVA-HAC2 were comparable, with most of the degradation occurring within the first week and full degradation occurring over the course of three weeks (FIG. 1D). To characterize in vivo degradation profile of OVA-encapsulated HAC1:Cy5 (OVAHAC1: Cy5) and OVA-encapsulated HAC2:Cy5 (OVA-HAC2:Cy5), in vivo imaging system (IVIS) was utilized. A single OVA-HAC1:Cy5 or OVA-HAC2:Cy5 was injected subcutaneously in the hind flank of C57Bl/6J (B6) mice. Strikingly, OVA-HAC1:Cy5 degraded over the course of 5 weeks whereas OVA-HAC2:Cy5 degraded over the course of 3 months (FIG. 1E and FIG. 1F). The degradation half-life of OVA-HAC1:Cy5 and OVA-HAC2:Cy5, as determined by the time to achieve a 50% reduction in fluorescence intensity was 20±2 and 54±5 days, respectively.

The effect of degradation on OVA release was then quantified. In vitro release assays for OVA-HAC1 and OVA-HAC2 were conducted with or without HYAL2 solution in phosphate buffered saline (PBS). In HYAL2 solution, 88.8±6.6% and 86.3±6.8% of OVA released from OVA-HAC1 and OVA-HAC2, respectively, over the first day with the remaining OVA released throughout the course of the study until the gels were fully degraded (FIG. 1G). In PBS, 22.2±2.8% and 23.0±0.7% of OVA burst release from OVA-HAC1 and OVA-HAC2, respectively, within the first day with minimal release over the rest of the study (FIG. 1G). The encapsulation efficiency of OVA, based on in vitro release, was 77.8% for OVA-HAC1 and 77.0% for OVA-HAC2. To determine in vivo OVA release kinetics, OVA was functionalized with Cy5 (OVA: Cy5) prior to encapsulation within HAC1 (OVA: Cy5-HAC1) and HAC2 (OVA: Cy5-HAC2) and measured using IVIS. Release was quantified by measuring the attenuation of the fluorescence signal relative to the initial measurement. The release of OVA:Cy5 from HAC1 and HAC2 was sustained over multiple weeks with accelerated OVA:Cy5 release from HAC1 as compared to HAC2 (FIG. 1H and FIG. 1I)

HAC1 and HAC2 are Infiltrated with a Distinct Innate Immune Cell Profile

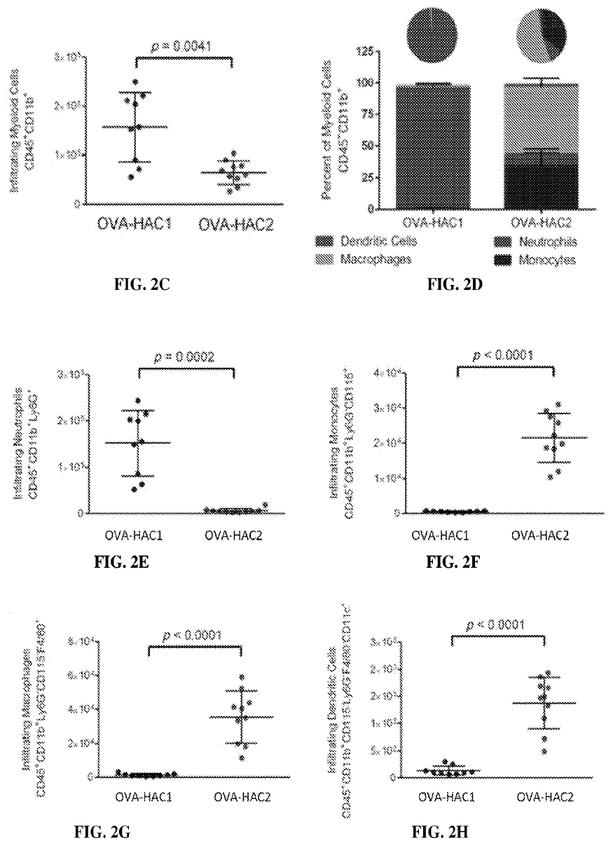

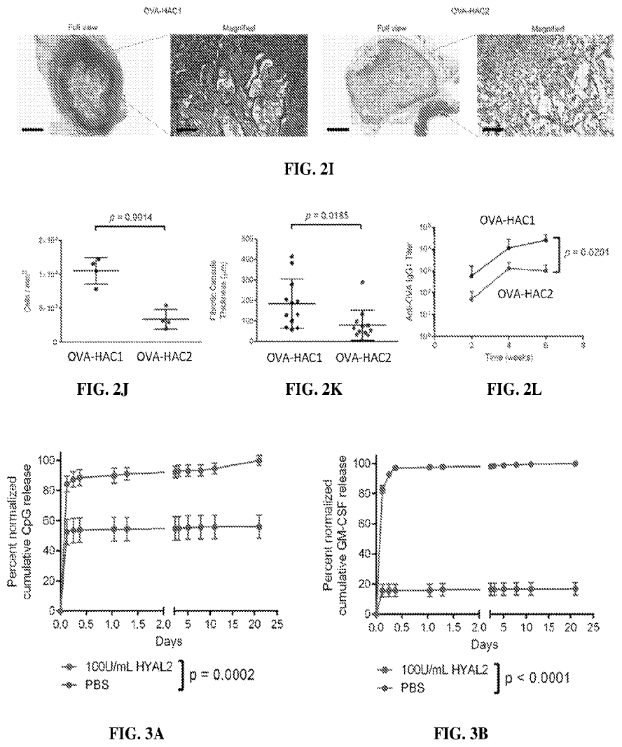

As endotoxin content of polymers is known to influence the immune response, the endotoxin content of HAC1 and HAC2 was quantified by measuring lipopolysaccharide (LPS) content. Endotoxin content was measured to be 7.3×10⁻³ EU and 6.7×10⁻⁴ EU for HAC1 and HAC2 respectively. Innate immune cells infiltrating OVA-HAC1 and OVA-HAC2 were assessed 7-days post-injection using flow cytometry (FIGS. 2A-B). The viability of infiltrating cells (Zombie Aqua™ 1 negative) was consistently greater than 95% in OVA-HAC1 and OVA-HAC2. Total CD45⁺CD11b⁺ (myeloid) cells were 2.4-fold higher in OVA-HAC1 compared to OVA-HAC2 (FIG. 2C). Notably, CD45⁺CD11b⁺ Ly6G⁺ (neutrophil) cells constituted most of the cellular infiltrates in OVA-HAC1 but were nearly absent in OVA-HAC2 (FIGS. 2D-E). Conversely, OVA-HAC1 had minimal CD45⁺CD11b⁺Ly6G⁻CD115⁺ (monocyte) and CD45⁺CD11b⁺Ly6G⁻CD115⁻F4/80⁺ (macrophage) cells whereas monocytes and macrophages constituted a majority of cellular infiltrates in OVA-HAC2 (FIGS. 2D, 2F, 2G). CD45⁺CD11b⁺Ly6G⁻CD115⁻F4/80⁻CD11c⁺ (dendritic) cells (DCs) were sparsely found in both OVA-HAC1 and OVA-HAC2 (less than 3% of total myeloid cells), but on average higher in OVA-HAC2 (FIGS. 2D, 2G).

Cryogels were explanted from mice 7-days post-injection for histomorphometric analysis using hematoxylin and eosin (H&E). Both OVA-HAC1 and OVA-HAC2 were infiltrated and encased in a fibrotic capsule (FIG. 2I). Cellularity and capsule thickness were increased in OVA-HAC1 (FIGS. 2J, 2K). In a separate cohort of mice, we measured the host immune response to OVA-HAC1 and OVA-HAC2. Mice received a single subcutaneous injection of OVA-HAC1 or OVA-HAC2 each in a prime and boost setting 11-days apart and were bled at pre-determined time points post-prime. OVA-HAC1 induced higher anti-OVA IgG1 antibody titers compared to OVA-HAC2 (FIG. 2L).

Next, whether the aforementioned differences in innate immune cell infiltration and anti-OVA antibody titers might be due to differences in endotoxin content was determined. LPS was added, the major constituent of endotoxin, in HA-Tz and Cy5-HA-Nb from to generate OVA-HAC2 with endotoxin content of 5.2×10⁻³ EU (low-LPS) and 5.2×10⁻² EU (high-LPS), corresponding to approximately 80% and 800% of OVA-HAC1 endotoxin content. Innate immune cell infiltration into OVA-HAC2 with LPS was assessed 7-days post-injection and compared to OVA-HAC2 without added LPS. Inclusion of LPS had no effect on total myeloid, neutrophil, monocyte, macrophage, or DC infiltration in OVA-HAC2. Mice were administered OVA-HAC2, low-LPS OVA-HAC2, and high-LPS OVA-HAC2 in a prime and boost setting 11-days apart and were bled at pre-determined time points. No significant differences were quantified with anti-OVA IgG1 antibody titers between any of the test groups. Based on these results, we selected HAC2 for further assessment.

### Encapsulation of Adjuvants Enhances HA Cryogel-Based Vaccine Efficacy and Alters Foreign Body Response.

To assess the effect of including an adjuvant in OVA-HAC2, we selected CpG-ODN 1826 (CpG, 100 microgram), a TLR9 agonist^{14,16} and granulocyte-colony stimulating factor (GM-CSF, 1 microgram), a DC maturation factor^{17,20} and alternative adjuvant^{21,23} were selected. CpG and GM-CSF were encapsulated in OVA-HAC2 (CpG-GMCSF-OVA-HAC2) and in vitro release studies were conducted with or without HYAL2 solution in PBS. In HYAL2 solution, 88.7±4.5% and 97.1±0.4% of CpG and GM-CSF, respectively, released from OVA-HAC2 over the first day with the remaining release throughout the course of the study with cryogel degradation (FIGS. 3A, 3B). In PBS, 53.8±7.1% and 15.8±3.6% of CpG and GM-CSF, respectively, burst release from the cryogels over the course of the first day with minimal release over the rest of the study (FIGS. 3A, 3B). From the release in PBS, encapsulation efficiency was calculated to be 46.2% and 84.2% for CpG and GM-CSF, respectively.

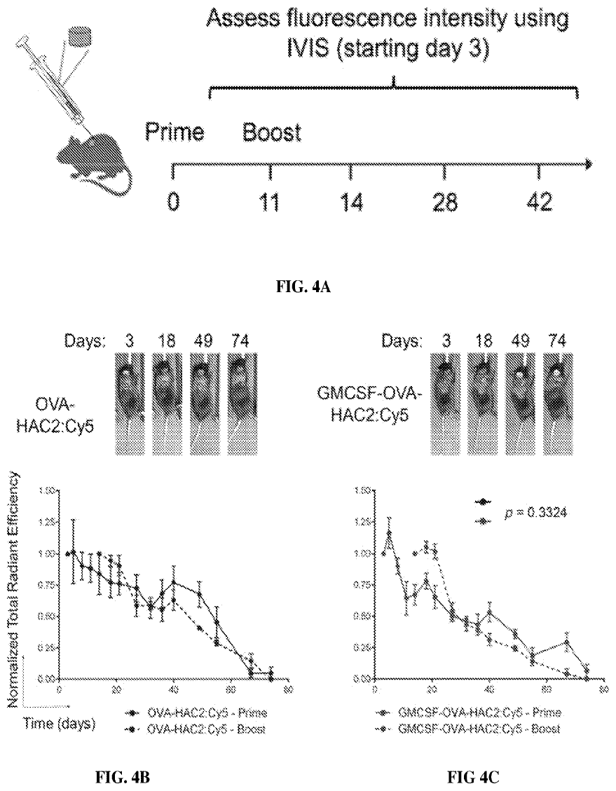

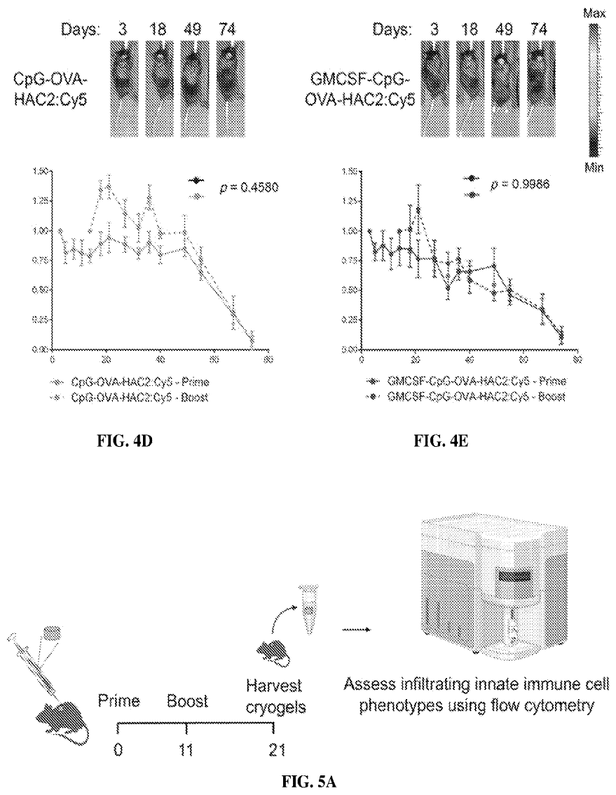

Three OVA-HAC2 adjuvanted formulations were made by inclusion of either CpG (CpG-OVA-HAC2), GM-CSF (GMCSF-OVA-HAC2), or both (GMCSF-CpG-OVA-HAC2) and degradation kinetics were assessed and compared to OVA-HAC2:Cy5 (FIG. 4A). All components were tested for endotoxin content. Mice were administered a prime and boost with adjuvanted OVA-HAC2:Cy5 formulations or OVA-HAC2:Cy5 11-days apart. Degradation profile was comparable between all groups and cryogels were fully degraded 10-weeks post-injection (FIGS. 4B-4E).

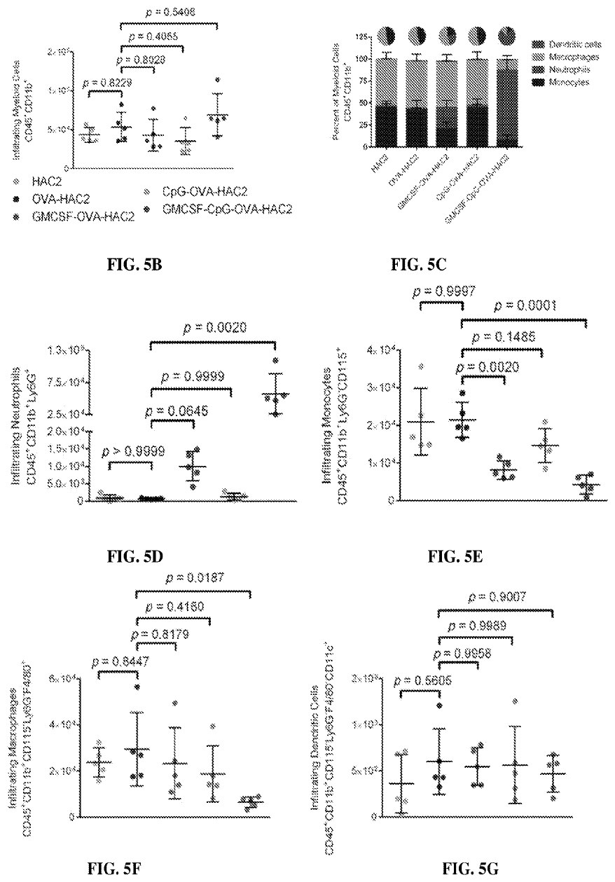

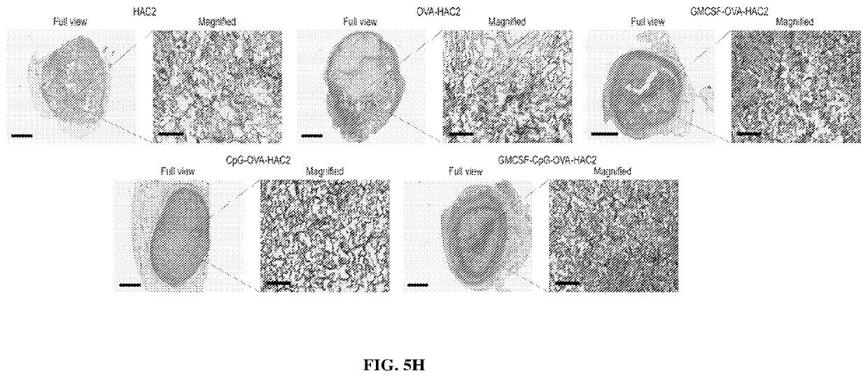

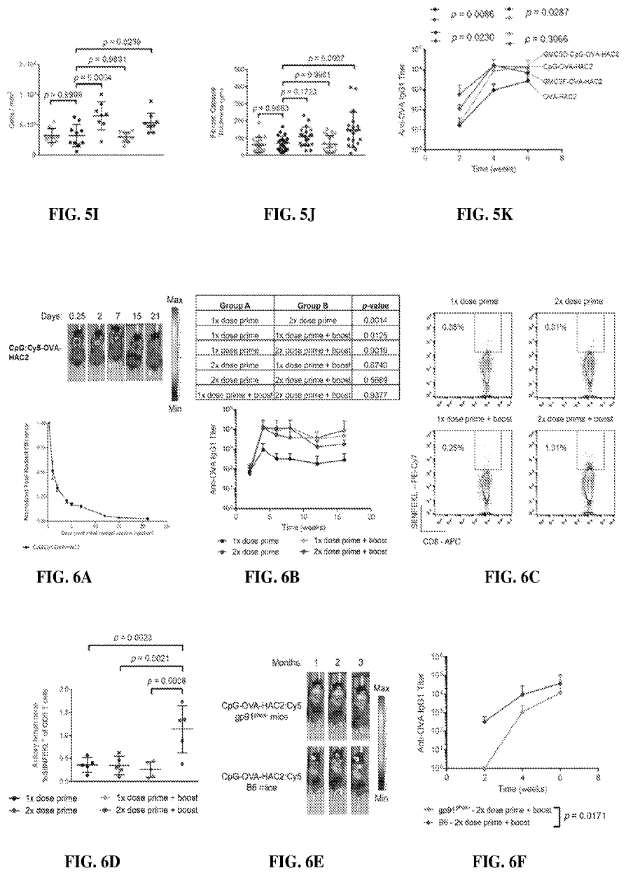

Innate immune cell infiltrates in the OVA-HAC2 vaccine formulations were compared 10-days and 21-days after injection (FIG. 5A). The viability of infiltrating cells was consistently greater than 93% in adjuvanted OVA-HAC2 and OVA-HAC2 10-days after injection. In cryogels removed 10-days after injection, total myeloid cell infiltration was similar between OVA-HAC2 and all adjuvanted OVA-HAC2 formulations (FIG. 5B). The fraction of Ly6G⁺ neutrophils was enhanced in both GMCSF-OVA-HAC2 and GMCSF-CpG-OVA-HAC2 and constituted the majority of infiltrating cells in GMCSF-CpG-OVA-HAC2 (FIG. 5C). Neutrophil cell counts were 14.5- and 83.2-fold higher in GMCSF-OVA-HAC2 and GMCSF-CpG-OVA-HAC2 respectively, compared to OVA-HAC2 (FIG. 5D). On the other hand, neutrophil count in CpG-OVA-HAC2 and OVA-HAC2 was similar (FIG. 5D). Monocytes were lower in all adjuvanted OVA-HAC2 formulations compared to OVA-HAC2. However, GMCSF-CpG-OVA-HAC2 had the lowest number of monocytes (FIG. 5E). Macrophage infiltration was modestly lower in GMCSF-CpG-OVA-HAC2, but similar in the other formulations (FIG. 5F). DC infiltration was similar between the test groups and constituted less than 3% of total cellular infiltrates in all formulations (FIGS. 5C, 5G). In the cryogel formulations removed 21-days after injection, live cell percentages were greater than 75% on average in all groups. Myeloid cell infiltration and the constituent cells of the infiltrates were similar between groups.

HAC2 vaccine formulations were administered in mice and removed 10-days post injection for histomorphometry-based H&E staining. All formulations were infiltrated with cells and encased in a fibrotic capsule (FIG. 5H). The cellularity was greater in HAC2 vaccine formulations which included GM-CSF as a constituent (FIG. 5I). However, only mice which received GMCSF-CpG-OVA-HAC2 had increased capsule thickness (FIG. 5J).

Initial immunization studies were conducted without HA in which mice received a single prime and boost bolus subcutaneous injection of vaccine components 11-days apart. Mice received either (i) OVA only, (ii) OVA+GM-CSF, (iii) OVA+CpG, or (iv) OVA+GM-CSF+CpG and periodically bled post-prime to assess anti-OVA IgG1 antibody concentrations. OVA+GM-CSF and OVA+GM-CSF+CpG enhanced antibody titers compared to OVA only. OVA+CpG generated a minimal antibody response, comparable to mice which received OVA only.

Next, the aforementioned OVA-HAC2 vaccine formulations were administered to mice in a prime and boost 11-days apart. Assessments of anti-OVA IgG1 antibody titers showed a significant difference between OVA-HAC2 and all other adjuvanted formulations (FIG. 5K). There was no difference in antibody titers between any adjuvanted OVA-HAC2 formulations (FIG. 5K). The same cohorts of mice were used to measure degradation of the OVA-HAC2:Cy5 formulations using IVIS. In a separate study, mice were administered prime and boost adjuvanted OVA-HAC2 and OVA-HAC2 only and sacrificed 3-weeks post prime to assess CD45+B220−CD8+SIINFEKL+ cells (OVA-specific cytotoxic T cell) in both the draining axillary lymph nodes (LNs) and spleen. The test groups and unvaccinated mice had comparable antigen-specific cytotoxic T cells in the lymph node and spleens.

It was concluded that all adjuvanted OVA-HAC2 vaccine formulations resulted in comparable enhanced antigen-specific adaptive immune response. However, formulations that included GM-CSF were associated with an exacerbated the foreign body response. Therefore, CpG-OVA-HAC2 was selected for further studies.

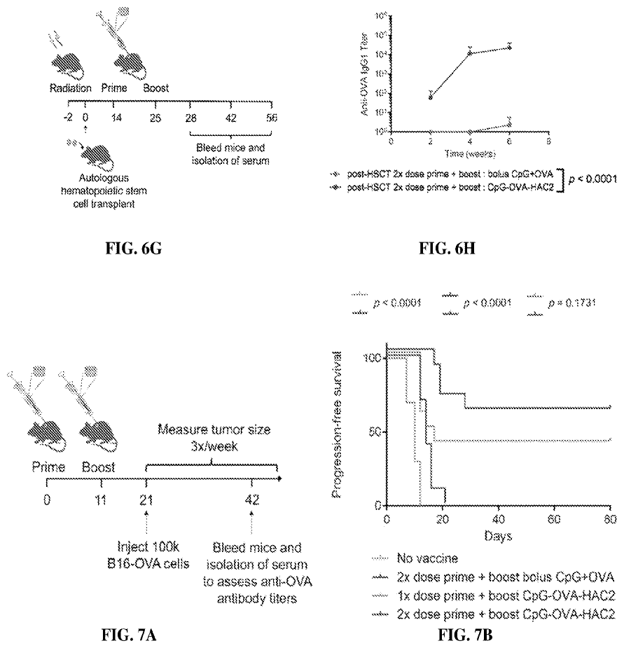

The release of CpG from CpG-OVA-HAC2 in vivo was evaluated. Cy5-labeled CpG (Cy5:CpG) was encapsulated within OVA-HAC2 to from CpG:Cy5-OVA-HAC2. Release was assessed using IVIS. CpG:Cy5 release was sustained over a period of three weeks with 85.0±3.1% of release occurring within the first week (FIG. 6A). The effect of vaccine dose escalation on the adaptive immune response was assessed by increasing the number of CpG-OVA-HAC2 administered. Mice received either a single dose prime, two-dose prime, single dose prime and boost, or two-dose prime and boost. Assessment of anti-OVA IgG1 antibody titers showed that a single prime vaccination induced significantly lower antibody titers than mice which received higher doses (FIG. 6B). Notably, there was no difference in anti-OVA IgG1 antibody titers in mice which received any of the other higher vaccine doses (FIG. 6B). CD45⁺B220⁻CD8⁺SIINFEKL⁺ cells (OVA-specific cytotoxic T cells) were assessed 3-weeks post-prime in the draining axillary lymph nodes (LNs) (FIG. 6C). Only mice which received two CpG-OVA-HAC2 as both a prime and boost had a significantly elevated percentage of OVA-specific cytotoxic T cells in axillary LN compared to other groups (FIG. 6D). There was no difference in OVA-specific cytotoxic T cell percentage in any of the groups in the spleen 3-weeks post prime.

To better study the effect of polymer degradation and the development of an adaptive immune response, gp91phox− mice were utilized, which have neutrophils that lack the ability to perform oxidative burst^{11,24,25}. It has also previously been demonstrated that HA cryogel degradation in this model is impaired¹¹. Two doses of CpG-OVA-HAC2:Cy5 were administered in gp91phox− or B6 mice as a prime and boost 11-days apart. IVIS measurements on mice showed minimal degradation of CpG-OVA-HAC2:Cy5 in gp91phox− mice and complete degradation in B6 mice (FIG. 6E). The same cohorts of mice were bled at pre-determined time-points for assessment of anti-OVA IgG1 antibody titers. There was a significant delay in the development of anti-OVA IgG1 antibodies in gp91phox− mice with none having detectable anti-OVA IgG1 antibody titers two weeks post prime (FIG. 6F).

The utility of CpG-OVA-HAC2 in mediating enhanced immunity, we measured the effect of treating immunodeficient mice post autologous hematopoietic stem cell transplant (HSCT) in B6 mice was measured. Mice received lethal radiation followed by HSCT consisting of both 15M whole bone marrow cells and 10M splenocytes 2-days later (FIG. 6G). Two CpG-OVA-HAC2:Cy5 or bolus CpG+OVA were administered to mice in a prime and boost 11-days apart and bled at pre-determined time-points (FIG. 6G). Mice that received CpG-OVA-HAC2 had measurable anti-OVA IgG1 antibody titers whereas there were no detectable anti-OVA IgG1 antibodies in mice which received bolus vaccination (FIG. 6H). The immunization study was repeated in the context of post-allogenic HSCT, with BALB/cJ donor mice and B6 recipients. All the mice in this study succumbed to graft versus host disease-like pathology within 5-weeks post-HSCT. Blood was collected once at 2-weeks post prime, 4-weeks post-HSCT. There were no detectable anti-OVA IgG1 antibodies in either group.

### CpG-OVA-HAC2 Mediates Prophylactic Protection in a B16-OVA Melanoma Mouse Model

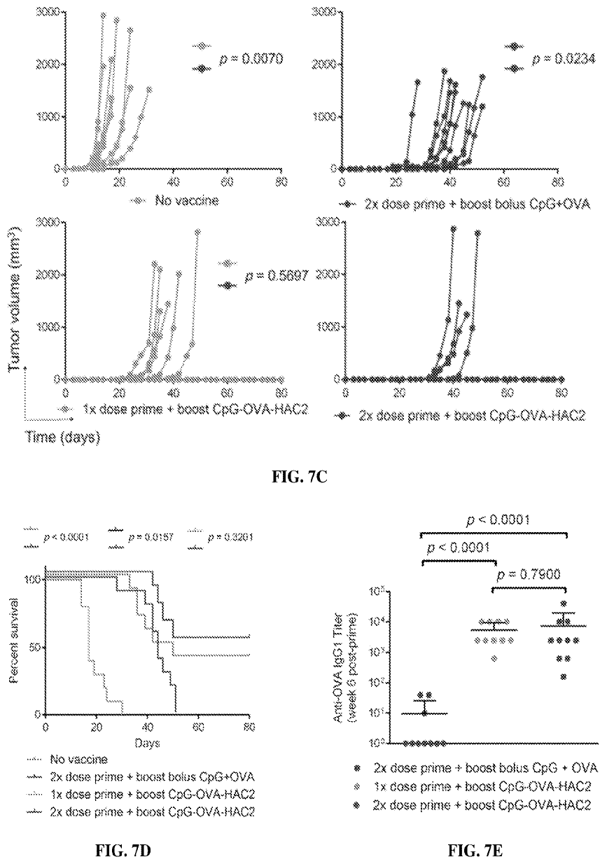

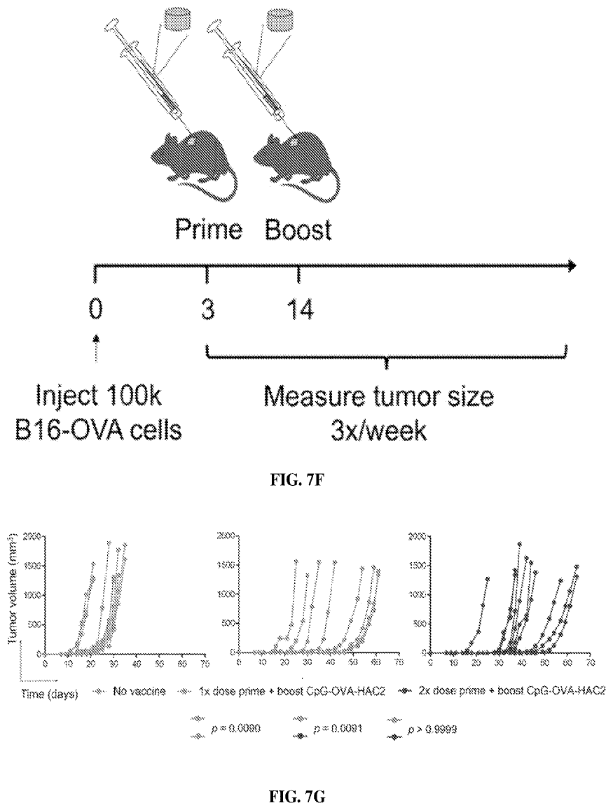

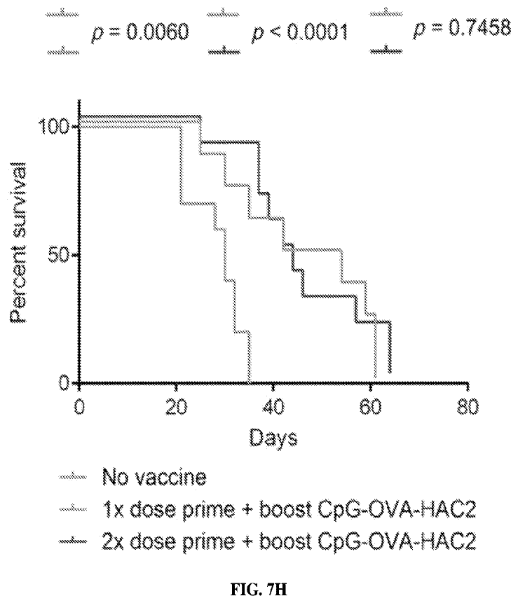

We sought to assess whether CpG-OVA-HAC2 enhanced prophylactic protection in a mouse melanoma model. Mice were administered either one or two CpG-OVA-HAC2 or two bolus CpG+OVA injections in a prime and boost setting. Mice received one hundred thousand B16-OVA cells administered subcutaneously in mice 3-weeks post prime or in unvaccinated mice and tumor growth rate and survival were compared (FIG. 7A). In all unvaccinated mice, tumors were visible two weeks post tumor inoculation with rapid tumor growth thereafter (FIGS. 7B, 7C). In mice that received two bolus CpG+OVA injections, tumor growth was delayed and were visible in all mice three weeks post tumor inoculation (FIG. 7B). In contrast, only 60% of the mice that received a single CpG-OVA-HAC2 as prime and boost had visible tumors. A total of 40% of the mice that received two CpG-OVA-HAC2 as prime and boost each had visible tumors four weeks post tumor inoculation (FIG. 7B). In all vaccinated mice, tumor growth rate was significantly reduced and increased the mean survival time (FIG. 7C). Overall survival was dose responsive to CpG-OVA-HAC2. Mice that received two CpG-OVA-HAC2 as a prime and boost had the highest overall survival of 60% (FIG. 7D). Mice that received a single CpG-OVA-HAC2 had an overall survival of 40% whereas mice receiving bolus vaccination and unvaccinated mice all succumb to B16-OVA melanoma (FIG. 7D). Vaccinated mice were bled six weeks post prime, three weeks post tumor inoculation, and mice which received bolus CpG-OVAHAC2 had lower anti-OVA IgG1 antibody titers than mice which received CpG-OVAHAC2 (FIG. 7E). Mice which survived B16-OVA melanoma challenge were re-challenged with 100K B16-OVA cells three months post initial inoculation. All mice survived re-challenge without visible tumors for at least 40 days post re-challenge.

It was next assessed whether CpG-AVA-HAC2 could provide therapeutic protection against murine melanoma. Mice were administered 100,000 B16-OVA cells subcutaneously 3-days prior to one or two CpG-OVA-HAC2 in a prime and boost setting (FIG. 7F). CpG-OVA-HAC2 administered therapeutically slowed tumor growth and prolonged survival as compared with unvaccinated mice (FIG. 7G and FIG. 7H). Mice that received a single CpG-OVA-HAC2 as a prime and boos survived 45.9±13.8 days and mice that received two CpG-OVA-HAC2 survived 45.5±12.0 days post-tumor challenge. In contrast, unvaccinated mice only survived 28.5±5.3 days post challenged (FIG. 7H).

### Discussion

Here, it was demonstrated that a hyaluronic acid cryogel-based biodegradable vaccine mediates sustained release of vaccine components, enhances antigen-specific adaptive immunity in healthy and immunodeficient mice, and provides protection against B16-OVA melanoma in a vaccine dose-dependent manner. The source of material and selection of adjuvants had a significant impact on the infiltrating innate immune cell subsets that constitute the FBR. We demonstrate that CpG-OVA-HAC2 formulation enhances anti-OVA adaptive immune response without significantly altering the FBR and that the adaptive immune response was enhanced with escalating vaccine doses. The effect of high-dose Cpg-OVA-HAC2 was studied in immune deficient models. In gp91-phox mice, the degradation of CpG-OVA-HAC2 was delayed which resulted in slowed activation of the adaptive immunity. In post-HSCT mice, adaptive immune activation with CpG-OVA-HAC2 was greatly enhanced as compared to bolus CpG+OVA vaccination, highlighting the enhancement in adaptive immune response even in immunodeficient contexts. Lastly, enhancement in immune response with increasing vaccine dose was assessed in prophylactic studies involving B16-OVA melanoma, in which the two-dose prime and boost vaccine dose provided greater protection than the single-dose prime and boost.

HA was selected as the polymer as it is ubiquitous in the ECM and has a long history of use as a biodegradable material to effectively facilitate sustained drug delivery. As an ECM component, HA provides cues to regulate inflammation and tissue repair²⁶. In the context of tissue damage or injury, HA undergoes degradation resulting in the production of low molecular weight fragments^{27,28}. These fragments are known to activate innate immune cells, particularly monocytes, macrophages, and DCs, through the activation of TLR2 and TLR4^{4,28,31}. The downstream signaling of TLR activation results in secretion of TNF-alpha, IL-12 and IL-1beta which promote inflammation^{32,33}. Additionally, HA fragments have been shown to induce expression of matrix metalloproteinases, which degrade ECM components and facilitate tissue remodeling^{34,35}. Thus, the use of HA in biomaterials based vaccine not only provides sustained release of vaccine components but also potentially offers additional cues to regulate inflammation. The use of HA is further supported by other pre-clinical vaccine studies which have utilized HA as an adjuvant in their vaccine formulations^{4,6}. Consistent with prior work, the instant results show that HA cryogel encapsulating OVA alone elicits anti-OVA IgG1 antibody titers without an additional adjuvant⁴.

Seeking to assess the effect of HA sourced from different sources, the effect of formulating HA cryogels from two commercial vendors was assessed. HA cryogels from both vendors had comparable pore size and morphology, in vitro degradation kinetics, and in vitro OVA release kinetics. There were differences in endotoxin levels, as determined by measuring LPS content, with HAC1 having 10.8-fold higher endotoxin levels than HAC2. However, the endotoxin levels of HAC1 and HAC2 were well below FDA guidelines for implantable devices^{36,37}. When cryogels were administered, OVA-HAC1 induced higher anti-OVA IgG1 titers than OVA-HAC2 but was associated with a neutrophil dominated FBR with enhanced capsule thickness. However, when we added external LPS into HAC2 such that the resulting endotoxin levels were 80% and 800% of those in HAC1, there were no significant differences in anti-OVA IgG1 antibody titers and FBR when compared to OVA-HAC2 without added LPS. These results suggest that increased LPS content alone in OVA-HAC1 cannot explain the differences in anti-OVA IgG1 antibody titers and FBR. These results suggest the potential of other components that constitute endotoxin which can also act as TLR agonists^{38,40}.

Adopting a strategy similar to other biomaterial vaccines, the addition of the adjuvants CpG and GM-CSF was tested^{1,2,8,41}. CpG is a clinically approved TLR9 agonist used in the Hepatitis B vaccines and has been extensively studied for use in numerous clinical vaccine formulations^{42,43}. GM-CSF is a cytokine that promotes myeloid cell development and in vitro DC maturation 172 which have been shown to accelerate post-HSCT bone marrow recovery⁴⁴. GM-CSF has also been studied extensively in pre-clinical vaccine formulations and has been used as an adjuvant unsuccessfully in several cancer vaccine clinical trials⁴⁵. In contrast to other common TLR agonists such as Monophosphoryl-Lipid A (MPLA) or squalene, CpG and GM-CSF are water soluble and amenable for encapsulation in HA cryogels. Avoiding the need for organic solvents, solubilizers and stabilizers reduces interference with the cross-linking process. If needed, encapsulating multiple TLR agonists, for example CpG and poly(I:C), may represent a strategy for improving immunogenicity of the HAC2-based vaccine formulation

In the instant studies, inclusion of either GM-CSF or CpG into OVA-HAC2 enhanced anti-OVA IgG1 antibody titers comparably and inclusion of both did not further enhance anti-OVA IgG1 antibody titers. When the innate immune cell infiltration in the various vaccine formulations, inclusion of GM-CSF lead to enhanced neutrophil infiltration, consistent with other reports. Supporting these results, analysis of H&E stained HAC2 vaccine formulations showed an increase in cellularity with formulations including GM-CSF. As the inclusion of GM-CSF in the vaccine increased inflammation at the injection site and did not further improve the immune response, CpG-OVA-HAC2 was chosen for further evaluation.

Previous findings of the importance of degradation in mediating release of encapsulated components¹¹ was further validated in the present work. In vitro release in PBS and HYAL2 solutions showed an initial burst release followed by a period of sustained release. Burst release in PBS is expected and is observed in nearly all cryogel-based drug delivery systems. The burst release in HYAL2 solution can be attributed to rapid initial degradation of the HA cryogels, 51.3±3.7% and 51.2±2.9% of gel degraded after first 9-hours for HAC1:Cy5 and HAC2:Cy5, respectively. In vivo, the rate of degradation of HA cryogels was substantially lower, the release kinetics for encapsulated vaccine components is more sustained.

CpG-OVA-HAC2 was administered in immunodeficient mice to assess the mechanism and potential enhancement of vaccine efficacy. Consistent with previous work, the degradation of HA cryogels in gp91phox− mice was impaired¹¹. Moreover, the induction of anti-OVA IgG1 antibody titers in the gp91phox− were significantly delayed, supporting the need for release of vaccine components. Post-HSCT Mice which received CpG-OVAHAC2 injections developed robust anti-OVA IgG1 antibody titers starting two-weeks post prime whereas mice which received bolus vaccination only developed failed to develop a detectable adaptive immune response until 6-weeks post-prime. These results support the utility of CpG-OVA-HAC2 in generating a robust adaptive immune response in immunodeficient mice.

Dose escalation studies with CpG-OVA-HAC2 showed both dose responsive behavior of anti-OVA IgG1 antibody titers and increased percentage of OVA-specific cytotoxic T cells of total cytotoxic T cells in draining axillary LNs. Anti-OVA IgG1 antibody titers plateaued at all doses higher than a single prime CpG-OVA-HAC2. Antibody plateau is commonly observed in other vaccine studies and is indicative of saturation of the humoral immune reposnse^{7,46,48}. There was no measurable expansion of OVA-specific cytotoxic T cells except when immunized with two-dose prime and boost, which was the highest vaccination dose. These findings are supported by the instant B16-OVA melanoma studies, in which mice that received CpG-OVA-HAC2 survived B16-OVA melanoma challenge, an effect that was dose-dependent. As protection against solid tumors is primarily mediated by antigen specific T cells^{49,50}, these studies support that antigen specific cytotoxic T cell expansion is vaccine dose dependent. These results are consistent with other vaccine formulations which show a dose-dependent effect in similar murine melanoma models^{49,51,52}.

The results from this study show that by sustaining release of vaccine components over the course of weeks, CpG-OVA-HAC2 induce a more potent adaptive immune response compared to conventional bolus vaccines. Taken together, our results demonstrate the development of a degradable, biocompatible vaccination platform which may be leveraged to provide greater protection against infections or cancers, including in settings of immunodeficiency.

### Methods

General methods and statistics. Sample sizes for animal studies were based on prior work without use of additional statistical estimations¹¹. Results were analyzed where indicated using student's t-test, one-way ANOVA with Dunnett's multiple comparison, Kruskal-Wallis test with Dunnet's multiple comparison, and two-way ANOVA with Bonferroni's test using Graphpad Prism software. Alphanumeric coding was used in blinding for pathology samples and cell counting.

Reagents. Sodium hyaluronate was purchased from Acros Organics (MW 1.5-2.2 MDa, lot: A0405554, Supplier 1) and NovaMatrix™ (MW 1.2-1.9 MDa, Pharma Grade 150, lot: 18011K, Supplier 2). (2-morpholinoethanesulfonic acid (MES), sodium chloride (NaCl), sodium hydroxide (NaOH), N-hydroxysuccinimide (NHS), 1-ethyl-3-(3-dimethylaminopropyl)-carbodiimide hydrochloride (EDC), sodium periodate (311448) and ammonia borane (AB) complex (682098) were purchased from Sigma-Aldrich. (4-(1,2,4,5-tetrzain-3-yl)phenyl)methanamine (tetrazine amine) was purchased from Kerafast™ (FCC659, lot: 2014). 1-bicyclo[2.2.1]hept-5-en-2-ylmethanamine (norbornene amine) was purchased from Matrix Scientific ($038023, lot: M15S). Cy5-tetrazine amine was purchased from Lumiprobe™ (lot: 9D2FH). 1 kDa molecular weight cutoff (MWCO) mPES membrane was purchased from Spectrum (S02-E001-05-N). GM-CSF was purchased from PeproTech™ (AF-315-03, lot: 081955). CpG (CpG ODN 1826, 5′-TCC ATG ACG TTC CTG ACG TT-3′ (SEQ ID NO: 1)) was purchased from Integrated DNA Technologies (lots: 480037977, 513470982). CpG:Cy5 comprises the same nucleotide sequence as CpG ODN 1826 but comprises a Cy5 label attached at the 5′ end and was purchased from Integrated DNA Technologies (lot: 526167013). Vaccine grade OVA was purchased from Invivogen (vac-pova-100, lot: 5822-04-01). Lipopolysaccharide (LPS) was purchased from Sigma-Aldrich (L3012-5MG, lot: 0000091258).

Derivatization of HA. Tetrazine functionalized HA (HA-Tz) or norbornene functionalized HA (HA-Nb) were prepared by reacting tetrazine amine or norbornene amine to HA using EDC/NHS carbodiimide chemistry. Sodium hyaluronate was dissolved in a buffer solution (0.75% wt/vol, pH~6.5) of 100 mM MES buffer. NHS and EDC were added to the mixture to activate the carboxylic acid groups on the HA backbone followed by either tetrazine amine or norbornene amine. HA from both suppliers was assumed to be 1.8 MDa for purposes of conjugation reactions. To synthesize HA-Tz, the molar ratios of HA:EDC:NHS:tetrazine are 1:25000:25000:2500. To synthesize HA-Nb, the molar ratios of HA:EDC:NHS:norbornene are 1:25000:25000:2500. Each reaction was stirred at room temperature for 24 hours and transferred to a 12,000 Da MW cutoff dialysis sack (Sigma Aldrich) and dialyzed in 4 L of NaCl solutions of decreasing molarity (0.125M, 0.100M, 0.075M, 0.050M, 0.025M, OM, OM, OM, OM) for 8 hours per solution. After dialysis, solutions containing HA-Tz or HA-Nb were frozen overnight and lyophilized (Labconco FreeZone™ 4.5) for 48 hours. Cy5 conjugated HA-Nb (Cy5-HA-Nb) was synthesized following a previously described technique¹¹. 0.8 mg of Cy5-Tz was reacted with 100 mg of HA-Nb at 0.2 wt/vol in DI water for 24 hours at 37° C. and purified by dialysis in DI water using a 12,000 Da MW cutoff dialysis sack for 48 hours. Dialysis water bath was changed every ~8 hours. The CyS-HA-Nb solution was then frozen overnight and lyophilized for 48 hours.

Cryogel formation. We followed a previously described cryogelation method¹¹. To form cryogels, aqueous solutions of 0.6% wt/vol HA-Tz and Cy5-HA-Nb were prepared by dissolving lyophilized polymers into deionized water and left on a rocker at room temperature for a minimum of 8 hours to allow for dissolution. The aqueous solutions were then pre-cooled to 4° C. before cross-linking to slow reaction kinetics. HA-Tz and HA-Nb solutions were mixed at a 1:1 volume ratio, pipetted into 30 microliter Teflon molds which were pre-cooled to −20° C., and quickly transferred to a −20° C. freezer to allow for overnight cryogelation. To form OVA-HA cryogels, HA-Tz and Cy5-HA-Nb were solubilized in 3.33 mg/mL OVA solution in DI water prior to cross-linking. To form HA cryogel vaccines, 1 microliter/cryogel of CpG (100 mg/mL concentration) and/or GM-CSF (1 mg/mL concentration) was added to HA-Tz or Cy5-HA-Nb solubilized in OVA solution prior to cross-linking.

Endotoxin Testing. Endotoxin testing was conducted using a commercially available endotoxin testing kit (88282, Thermo Fisher Scientific, lot: VH310729) and following manufacturer's instructions. HA-Tz and Cy5-HA-Nb from both suppliers were solubilized at 0.3 wt % in endotoxin free water and samples were tested in technical triplicates. To calculate endotoxin content of a single HA cryogel, the EU/mL concentration for HA-Tz and Cy5-HA-Nb were multiplied by 0.03 (30 uL of volume per HA cryogel). Other components of the HA cryogel vaccine formulations tested for endotoxin included CpG (100 microgram/mL concentration), OVA (100 microgram/mL concentration), GM-CSF (1 microgram/mL concentration), and RO water which was used to solubilize all vaccine components.

Pore size analysis of HA cryogels. For scanning electron microscopy (SEM), frozen HA cryogels were lyophilized for 24 hours in their molds. Lyophilized HA cryogels were adhered onto sample stubs using carbon tape and coated with iridium in a sputter coater. Samples were imaged using secondary electron detection on a FEI Quanta 250 field emission SEM in the Nano3 user facility at UC San Diego. Fluorescence images of Cy5-labeled HA cryogels were acquired using a Leica SP8. All experiments were performed at the UC San Diego School of Medicine Microscopy Core. Pore size quantification of SEM images was quantified using FIJI image processing package⁵³.

LPS doping of HAC2: LPS was added to HA-Tz and HA-Nb solutions from supplier 2 at either 12.5 ng/100 mg or 1.25 ng/100 mg. The mixtures were stirred for 24 hours and then frozen overnight and lyophilized for 48 hours. Lyophilized polymers were rehydrated to assess endotoxin concentration prior to conducting in vivo studies.

HA cryogel pore-interconnectedness analysis. Cy5-labeled HA cryogels were incubated in 1 mL of FITC-labeled 10 μM diameter melamine resin micro particles (Sigma Aldrich) at 0.29 mg/mL concentration on a rocker at room temperature overnight. Fluorescence images of HA cryogels with FITC-labeled microparticles were acquired using a Leica SP8 confocal. Interconnectedness of the HA cryogels was determined by generating 3D renderings of confocal z-stacks using FIJI imaging processing package and assessing fluorescence intensity of both the Cy5 and FITC channels with depth starting from the top of the HA cryogel. All experiments were performed at the UC San Diego School of Medicine Microscopy Core.

In vitro degradation of HA cryogels. Cy5-labeled HA cryogels were placed into individual 1.5 mL microcentrifuge tubes (3448, Thermo Scientific, lot: 20430852) with 1 mL of 100 U/mL Hyaluronidase from sheep testes Type II (HYAL2, H2126, Sigma Aldrich, lot: SLBZ9984) in 1× phosphate buffered saline (PBS). Degradation studies were conducted in tissue culture incubators at 37° C. Supernatant from samples were collected by centrifuging the samples at room temperature at 2,000G for 5 minutes and removing 0.9 mL of supernatant. HA cryogels were resuspended by adding 0.9 mL of freshly made 100 U/mL HYAL2 in 1×PBS. Fluorescence measurements were conducted using a NanoDrop™ 2000 Spectrophotometer (Thermo Fisher Scientific) and these values were normalized to sum of the fluorescence values over the course of the experiment.

In vitro release assays. OVA-HAC1, OVA-HAC2, CpG-OVA-HAC2, or GMCSF-OVAHAC2 were placed into individual 1.5 mL microcentrifuge tubes in either 1 mL of 1×PBS or 100 U/mL HYAL2 solubilized in 1×PBS. Quantification of in vitro OVA release was conducted using a Micro BCA kit (Thermo Fisher Scientific, lot: UD277184) on supernatant samples. Absorbance values of 100 U/mL HYAL2 solution was assessed and subtracted from absorbance values in supernatant containing HYAL2 solution. Quantification of in vitro CpG release was conducted using OliGreen Assay kit (Thermo Fisher Scientific, lot: 2471822). Quantification of in vitro GM-CSF release was conducted using Murine GM-CSF ELISA kit (PeproTech™, lot: 1010055).

Cell lines and cell culture. The B16-OVA cell line was obtained from Professor Liangfang Zhang's laboratory (University of California, San Diego). The cells were cultured in Dulbecco's modified eagle medium (DMEM, Gibco, lot: 2060444) supplemented with 10% fetal bovine serum (Gibco), 1% penicillin-streptomycin (Corning, lot: 30002357), and 300 microgram/mL hygromycin B (Corning, lot: 30240136, selective antibiotic for OVA-expressing cells).

In vivo mouse experiments. All animal work was conducted at the Moores Cancer Center vivarium at UC San Diego and approved by the Institutional Animal Care and Use Committee (IACUC). All animal experiments followed the National Institutes of Health guidelines and relevant AALAC-approved procedures. Female C57BL/6J (B6, Jax #000664) and BalbC (Jack #000651) were 6-8 weeks at the start of the experiments. Male B6 and B6.129S-Cybb^{tm1Din} (gp91phox−, Jax #002365) mice were 6-8 weeks old at the start of experiments. All mice in each experiment were age matched and no randomization was performed.

Subcutaneous cryogel administration: HA cryogels suspended in 200 microliter of sterile 1×PBS were administered into the dorsal flank of mice by means of a 16G needle positioned approximately midway between the hind- and forelimbs. The site of injection was shaved and wiped with a sterile alcohol pad prior to gel injection.

In vivo cryogel degradation. In vivo OVA-HAC1:Cy5 and OVA-HAC2:Cy5 degradation studies were performed in B6 mice and gp91phox− mice. In all cases, Cy5-labeled cryogels were administered into the dorsal flank of an anesthetized mouse and the fluorescent intensity of the HA cryogel was quantified using an IVIS spectrometer (PerkinElmer) at predetermined time-points and analyzed using LivingImage™ software (PerkinElmer). At each time-point, mice were anesthetized and the area around the subcutaneous cryogel was shaved to reduce fluorescence signal attenuation. Fluorescence radiant efficiency, the ratio of fluorescence emission to excitation, was measured longitudinally as a metric to quantify fluorescence from subcutaneous cryogels. These values were normalized to the measured signal on day 3. All experiments were performed at the Moores Cancer Microscopy Core Facility at UC San Diego Health using an IVIS spectrometer.

In vivo release. For in vivo release assays, OVA:Cy5 was prepared by reacting OVA with sulfo-Cy5 NHS ester (Lumiprobe, lot: lot: 7FM7C) at a 1:50:5 molar ratio of OVA:EDC:Sulfo-Cy5 NHS ester in MES buffer to form OVA:Cy5. Unreacted EDC and sulfo-Cy5 NHS ester were removed by overnight dialysis using a 12 kDa MWCO dialysis membrane (D6191, Sigma Aldrich, lot: SLCL5005). 100 microgram of OVA:Cy5 was added to either HAC1 or HAC2 polymer mixture prior to cross-linking. To make CpG:Cy5-OVAHAC2, 100 microgram of CpG:Cy5 was added to OVA-HAC2 polymer mixture prior to cross-linking. For all in vivo release assays, release was assessed normalized to a 6-hour initial time-point in an analogous manner to in the in vivo cryogel degradation studies in the previous section.

Detection of serum anti-OVA IgG1 antibody titers. Blood was first collected from the tail vein of mice into EDTA coated tubes (365974, BD, lot: 2181885). Blood was centrifuged at 2,000G for 10 minutes at room temperature for serum collection. Anti-OVA IgG1 antibody titers were quantified using ELISA following established protocol⁴¹. High binding (3590, Corning) ELISA plates were coated with 1 microgram/mL OVA in PBS at 4° C. overnight. Serum samples were diluted ranging from 1:8 to 1:1:163840 and incubated with the plates at room temperature for 1.5 hours before staining for mouse IgG1 (406604, Biolegend, lot: B270354). The anti-OVA titer was defied as the lowest serum dilution with an optical density value above 0.2

Flow cytometry analysis. Anti-mouse antibodies to CD45 (30-F11, lot: B280746), CD11b (M1/70, lot: B322056), Ly6-G/Gr-1 (1A8, lot: B259670), CD115 (CSF-1R, lot: B291837), CD11c (N418, lot: B346713), CD4 (RM4-5, lot: B240051), CD8″ (53-6.7, lot: B266721), and B220 (RA3-6B2, lot: B298555) were purchased from Biolegend. Anti-mouse F4/80 (BM8, lot: 2229150) and was purchased from eBioscience. SIINFEKL tetramer (SEQ ID NO: 13) (lot: 57396) was sourced from the NIH tetramer core. All cells were gated based on forward and side scatter characteristics to limit debris, including dead cells. Zombie Aqua™ fixable viability kit (423102, Biolegend, lot: B348291) was used to separate live and dead cells. Antibodies were diluted 1:400 v/v in staining buffer in and added to cells in 1:1 v/v ratio. Cells were gated based on fluorescence-minus-one controls, and the frequencies of cells staining positive for each marker were recorded. To quantify infiltrating immune cells within HA cryogels, spleen, and axillary LNs, mice were sacrificed, organ or HA cryogel was removed, and crushed against a 70-micron filter screen before antibody staining. All flow cytometry experiments were performed using a Attune® NxT Acoustic Focusing cytometer analyzer (A24858) at UC San Diego. Flow cytometry was analyzed using FlowJo (BD) software.

Histology. After euthanasia, HA cryogels were explanted and fixed in 4% paraformaldehyde (PFA) for 24 hours. 4% PFA was prepared by diluting 16% PFA stock (28908, Thermo Fisher Scientific, lot: XB340632) in 1×PBS. The fixed HA cryogels were then transferred to 70% ethanol solution. Samples were routinely processed, and sections (5 micron) were stained and digitized using an Aperio AT2 Automated Digital Whole Slide Scanner by the Tissue Technology Shared Resource at the Moores Cancer Center at UC San Diego Health. Digital slides were rendered in QuPath and positive cell detection was used to quantify the total number of mononuclear cells within each image. Quantification of mononuclear cell density was determined for each histological section. To quantify fibrotic capsule thickness, QuPath was used to measure the epithelial cell layer starting at the edge of the cryogel. Four measurements per cryogel were taken and all measurements were pooled for analysis.

Hematopoietic stem cell transplant. Irradiations were performed with a Cesium-137 gamma-radiation source irradiator (J.L. Shepherd & Co.). Syngeneic HSCT (B6 donor and recipient) and allogenic HSCT (BalbC donors, B6 recipients) consisted of 2 doses of 5000 cGy 6-hours apart+15M whole bone marrow cells and 10M splenocytes. Bone marrow cells for transplantation (from donors) were harvested by crushing all limbs with a mortar and pestle, diluted in 1×PBS, filtering the tissue homogenate through a 70 micron mesh and preparing a single-cell suspension by passing the cells in the flow through once through a 20-gauge needle. Splenocytes were collected by crushing spleens against a 70 micron mesh and preparing a single-cell suspension by passing the cells in the flow through once through a 20-gauge needle. Total cellularity was determined by counting cells using a hemacytometer. Subsequently, cells were suspended in 100 microliter of sterile 1×PBS and administered to anesthetized mice via a single retroorbital injection. All experiments were performed at the Moores Cancer Animal Facility at UC San Diego Health.

Prophylactic immunization. CpG-OVA-HAC2 were administered to mice in a prime and boost schedule 11-days apart. Unvaccinated mice were used as a control. Mice were challenged 21 days post-prime with 100 thousand B16-OVA cells in 100 microliter cold PBS injected subcutaneously below the neck using an insulin syringe. Tumor growth was monitored using calipers. Tumor volume was calculated assuming an ellipsoid (Volume=4/3*<*long axis/2*short axis/2*height/2). Mice were euthanized when the tumor volume exceeded 1200 mm³.

### REFERENCES

1. Roth, G. A. et al. Injectable Hydrogels for Sustained Codelivery of Subunit Vaccines Enhance Humoral Immunity. ACS Cent Sci 6, 1800.1812 (2020).
2. Shah, N. J. et al. A biomaterial-based vaccine eliciting durable tumour-specific responses against acute myeloid leukaemia. Nat Biomed Eng 4, 40.51 (2020).
3. Cirelli, K. M. et al. Slow Delivery Immunization Enhances HIV Neutralizing Antibody and Germinal Center Responses via Modulation of Immunodominance. Cell 177, 1153-1171.e28 (2019).
4. Dalla Pieti, A. et al. Hyaluronan is a natural and effective immunological adjuvant for protein-based vaccines. Cell Mol Immunol 18, 1197.1210 (2021).
5. Chen, J. et al. Sustained Delivery of SARS-CoV-2 RBD Subunit Vaccine Using a High Affinity Injectable Hydrogel Scaffold. Adv Healthc Mater 11, 2101714 (2022).
6. Moon, S., Shin, E.-C., Noh, Y.-W. & Lim, Y. T. Evaluation of hyaluronic acid based combination adjuvant containing monophosphoryl lipid A and aluminum salt for hepatitis B vaccine. Vaccine 33, 4762.4769 (2015).
7. Kuai, R. et al. Dual TLR agonist nanodiscs as a strong adjuvant system for vaccines and immunotherapy. Journal of Controlled Release 282, 131.139 (2018).
8. Shih, T.-Y. et al. Injectable, Tough Alginate Cryogels as Cancer Vaccines. Adv Healthc Mater 7, 1701469 (2018).
9. Weiden, J. et al. Robust Antigen-Specific T Cell Activation within Injectable 3D Synthetic Nanovaccine Depots. ACS Biomater Sci Eng 7, 5622.5632 (2021).
10. Selders, G. S., Fetz, A. E., Radic, M. Z. & Bowlin, G. L. An overview of the role of neutrophils in innate immunity, inflammation and host-biomaterial integration. Regen Biomater 4, 55.68 (2017).
11. Kerr, M. D. et al. Immune-responsive biodegradable scaffolds for enhancing neutrophil regeneration. Bioeng Transl Med 8, (2023).
12. Burdick, J. A., Chung, C., Jia, X., Randolph, M. A. & Langer, R. Controlled Degradation and Mechanical Behavior of Photopolymerized Hyaluronic Acid Networks. Biomacromolecules 6, 386.391 (2005).
13. Hopkins, K. et al. Sustained degradation of hyaluronic acid using an in situ forming implant. PNAS Nexus 1, pgac193 (2022).
14. Weeratna, R. D., Makinen, S. R., McCluskie, M. J. & Davis, H. L. TLR agonists as vaccine adjuvants: comparison of CpG ODN and Resiquimod (R-848). Vaccine 23, 5263.5270 (2005).
15. Zhang, X. et al. The Toll-Like Receptor 9 Agonist, CpG-Oligodeoxynucleotide 1826, Ameliorates Cardiac Dysfunction After Trauma-Hemorrhage. Shock 38, (2012).
16. Krogmann, A. O. et al. Proinflammatory Stimulation of Toll-Like Receptor 9 with High Dose CpG ODN 1826 Impairs Endothelial Regeneration and Promotes Atherosclerosis in Mice. PLoS One 11, e0146326- (2016).
17. Inaba, K. et al. Identification of proliferating dendritic cell precursors in mouse blood. Journal of Experimental Medicine 175, 1157.1167 (1992).
18. Sallusto, F. & Lanzavecchia, A. Efficient presentation of soluble antigen by cultured human dendritic cells is maintained by granulocyte/macrophage colony stimulating factor plus interleukin 4 and downregulated by tumor necrosis factor alpha. Journal of Experimental Medicine 179, 1109.1118 (1994).
19. Lutz, M. B. et al. Immature dendritic cells generated with low doses of GM-CSF in the absence of IL-4 are maturation resistant and prolong allograft survival in vivo. Eur J Immunol 30, 1813.1822 (2000).
20. Zhan, Y., Lew, A. M. & Chopin, M. The Pleiotropic Effects of the GM-CSF Rheostat on Myeloid Cell Differentiation and Function: More Than a Numbers Game. Front Immunol 10, (2019).
21. Zhang, C., Wang, B. & Wang, M. GM-CSF and IL-2 as adjuvant enhance the immune effect of protein vaccine against foot-and-mouth disease. Virol J 8, 7 (2011).
22. Hasan, M. S. et al. Granulocyte Macrophage Colony-Stimulating Factor as an Adjuvant for Hepatitis B Vaccination of Healthy Adults. J Infect Dis 180, 2023. 2026 (1999).
23. Zhao, W., Zhao, G. & Wang, B. Revisiting GM-CSF as an adjuvant for therapeutic vaccines. Cell Mol Immunol 15, 187.189 (2018).
24. Banerjee, E. R. & Henderson, W. R. Role of T cells in a gp91phox knockout murine model of acute allergic asthma. Allergy, Asthma & Clinical Immunology 9, 6 (2013).
25. Pollock, J. D. et al. Mouse model of X.linked chronic granulomatous disease, an inherited defect in phagocyte superoxide production. Nat Genet 9, 202.209 (1995).
26. Petrey, A. & de la Motte, C. Hyaluronan, a Crucial Regulator of Inflammation. Front Immunol 5, (2014).
27. Ruppert, S. M., Hawn, T. R., Arrigoni, A., Wight, T. N. & Bollyky, P. L. Tissue integrity signals communicated by high-molecular weight hyaluronan and the resolution of inflammation. Immunol Res 58, 186.192 (2014).
28. Gariboldi, S. et al. Low Molecular Weight Hyaluronic Acid Increases the Self-Defense of Skin Epithelium by Induction of #-Defensin 2 via TLR2 and TLR4. The Journal of Immunology 181, 2103.2110 (2008).
29. Rayahin, J. E., Buhrman, J. S., Zhang, Y., Koh, T. J. & Gemeinhart, R. A. High and Low Molecular Weight Hyaluronic Acid Differentially Influence Macrophage Activation. ACS Biomater Sci Eng 1, 481.493 (2015).
30. Bourguignon, L. Y. W., Wong, G., Earle, C. A. & Xia, W. Interaction of low molecular weight hyaluronan with CD44 and toll-like receptors promotes the actin filament-associated protein 110-actin binding and MyD88-NF>B signaling leading to proinflammatory cytokine/chemokine production and breast tumor invasion. Cytoskeleton 68, 671.693 (2011).
31. Romo, M. et al. Small fragments of hyaluronan are increased in individuals with obesity and contribute to low-grade inflammation through TLR-mediated activation of innate immune cells. Int J Obes 46, 1960.1969 (2022).
32. Kim, H. S. & Chung, D. H. TLR4-mediated IL-12 production enhances IFN-? and IL-1# production, which inhibits TGF-# production and promotes antibody-induced joint inflammation. Arthritis Res Ther 14, R210 (2012).
33. Park, S. H., Choi, H.-J., Lee, S. Y. & Han, J.-S. TLR4-mediated IRAK1 activation induces TNF-″ expression via JNK-dependent NF->B activation in human bronchial epithelial cells. Eur J Inflamm 13, 183.195 (2015).
34. Isnard, N., Legeais, J.-M., Renard, G. & Robert, L. EFFECT OF HYALURONAN ON MMP EXPRESSION AND ACTIVATION. Cell Biol Int 25, 735.739 (2001).
35. Shirvaikar, N., Montano, J., Turner, A. R., Ratajczak, M. Z. & Janowska-Wieczorek, A. Upregulation of MT1-MMP Expression by Hyaluronic Acid Enhances Homing-Related Responses of Hematopoietic CD34+ Cells to an SDF-1 Gradient. Blood 104, 2889 (2004).
36. Guidance for Industry Pyrogen and Endotoxins Testing: Questions and Answers. Food and Drug Administration 1.13 Preprint at https://www.fda.gov/regulatoryinformation/search-fda-guidance-documents/guidance-industry-pyrogen-andendotoxins-testing-questions-and-answers (2012).
37. Gorbet, M. B. & Sefton, M. V. Endotoxin: The uninvited guest. Biomaterials 26, 6811.6817 (2005).
38. Sato, M. et al. Direct Binding of Toll-Like Receptor 2 to Zymosan, and Zymosan-Induced NF->B Activation and TNF-″ Secretion Are Down-Regulated by Lung Collectin Surfactant Protein A1. The Journal of Immunology 171, 417.425 (2003).
39. Ma, P., Wang, Z., Pflugfelder, S. C. & Li, D.-Q. Toll-like receptors mediate induction of peptidoglycan recognition proteins in human corneal epithelial cells. Exp Eye Res 90, 130.136 (2010).
40. Amemiya, K. et al. Activation of Toll-Like Receptors by Live Gram-Negative Bacterial Pathogens Reveals Mitigation of TLR4 Responses and Activation of TLR5 by Flagella. Front Cell Infect Microbiol 11, (2021).
41. Najibi, A. J., Shih, T.-Y. & Mooney, D. J. Cryogel vaccines effectively induce immune responses independent of proximity to the draining lymph nodes. Biomaterials 281, 121329 (2022).
42. Kwon, J. Y., Daoud, N. D., Hashash, J. G., Picco, M. F. & Farraye, F. A. Efficacy of Hepatitis B Vaccination with a Novel Immunostimulatory Sequence Adjuvant (Heplisav-B) in Patients With Inflammatory Bowel Disease. Inflamm Bowel Dis 29, 254.259 (2023).
43. Scheiermann, J. & Klinman, D. M. Clinical evaluation of CpG oligonucleotides as adjuvants for vaccines targeting infectious diseases and cancer. Vaccine 32, 6377.6389 (2014).
44. Advani, R. et al. Granulocyte-Macrophage Colony-Stimulating Factor (GM-CSF) as an Adjunct to Autologous Hemopoietic Stem Cell Transplantation for Lymphoma. Ann Intern Med 116, 183.189 (1992).
45. Kerr, M. D., McBride, D. A., Chumber, A. K. & Shah, N. J. Combining therapeutic vaccines with chemo- and immunotherapies in the treatment of cancer. Expert Opin Drug Discov 16, (2021).
46. Vogel, A. B. et al. BNT162b vaccines protect rhesus macaques from SARS-CoV-2. Nature 592, 283.289 (2021).
47. Levi, R. et al. One dose of SARS-CoV-2 vaccine exponentially increases antibodies in individuals who have recovered from symptomatic COVID-19. J Clin Invest 131, (2021).
48. Rhodes, S. J., Knight, G. M., Kirschner, D. E., White, R. G. & Evans, T. G. Dose finding for new vaccines: The role for immunostimulation/immunodynamic modelling. J Theor Biol 465, 51.55 (2019).
49. Bencherif, S. A. et al. Injectable cryogel-based whole-cell cancer vaccines. Nat Commun 6, 7556 (2015).
50. Zhang, Z. et al. T Cell Dysfunction and Exhaustion in Cancer. Front Cell Dev Biol 8, (2020).
51. Fusciello, M. et al. Artificially cloaked viral nanovaccine for cancer immunotherapy. Nat Commun 10, 5747 (2019).
52. Chiu, Y.-C., Gammon, J. M., Andorko, J. I., Tostanoski, L. H. & Jewell, C. M. Modular Vaccine Design Using Carrier-Free Capsules Assembled from Polyionic Immune Signals. ACS Biomater Sci Eng 1, 1200.1205 (2015).
53. Schindelin, J. et al. Fiji: an open-source platform for biological-image analysis. Nat Methods 9, 676.682 (2012).

What is claimed is:

1. A sustained release vaccine composition, comprising:
(a) a biodegradable scaffold;
(b) an antigen; and
(c) an adjuvant;
wherein the antigen and adjuvant are disposed within the biodegradable scaffold, and wherein degradation of the biodegradable scaffold results in release of the antigen and the adjuvant from the biodegradable scaffold.

2. The sustained release vaccine composition of claim 1, wherein the antigen and adjuvant are released in a burst phase and a sustained release phase.

3. The sustained release vaccine composition of claim 1, wherein the biodegradable scaffold is comprised of hyaluronic acid polymers.

4. The sustained release vaccine composition of claim 3, wherein the hyaluronic acid polymers have a molecular weight of about 0.1 MDa to about 10 MDa.

5. The sustained release vaccine composition of claim 1, wherein the biodegradable scaffold is porous and has an average pore size of from about 20 micron to about 200 micron.

6. The sustained release vaccine composition of claim 1, wherein the biodegradable scaffold is a hydrogel.

7. The sustained release vaccine composition of claim 1, wherein the adjuvant is a TLR9 agonist.

8. The sustained release vaccine composition of claim 7, wherein the TLR9 agonist is a CpG oligonucleotide.

9. The sustained release vaccine composition of claim 1, wherein the adjuvant is a dendritic cell maturation factor.

10. The sustained release vaccine composition of claim 9, wherein the dendritic cell maturation factor GM-CSF.

11. The sustained release vaccine composition of claim 1, wherein the antigen is a tumor-associated antigen.

12. The sustained release vaccine composition of claim 1, wherein the antigen is a pathogen-associated antigen.

13. The sustained release vaccine composition of claim 1, wherein the composition has a degradation-half life in vivo of at least two weeks.

14. The sustained release vaccine composition of claim 1, wherein the composition releases the antigen and/or the adjuvant in vivo for a period of at least one week.

15. The sustained release vaccine composition of claim 1, wherein the composition is infiltrated by one or more immune cells following administration to a subject.

16. The sustained release vaccine composition of claim 15, wherein the one or more immune cells comprise a lower proportion of monocytes compared to a corresponding composition without the adjuvant.

17. The sustained release vaccine composition of claim 1, wherein the composition does not elicit inflammation associated with foreign body response upon administration to a subject.

18. The sustained release vaccine composition of claim 1, wherein the composition elicits a greater antibody response upon administration to a subject compared to a corresponding composition without the adjuvant.

19. A pharmaceutical composition comprising the sustained release vaccine composition of claim 1 and a pharmaceutically acceptable carrier.

20. The pharmaceutical composition of claim 19, wherein the pharmaceutical composition is formulated for subcutaneous administration.

21. A method of performing a vaccination in a subject, the method comprising administering to the subject the sustained release vaccine composition of claim 1.

22. The method of claim 21, wherein the sustained release vaccine composition is administered subcutaneously.

23. The method of claim 21, wherein the administering the sustained release vaccine composition comprises administering a prime and a boost dose of the sustained release vaccine composition.

24. A method of preparing a sustained release vaccine composition, comprising: crosslinking at a temperature below 0° C. a plurality of biodegradable polymers in the presence of an antigen and an adjuvant to provide a biodegradable scaffold with the antigen and adjuvant disposed therein.

25. The method of claim 24, wherein the biodegradable scaffold forms a hydrogel.

26. The method of claim 24, wherein the biodegradable polymers comprise hyaluronic acid polymers.

27. The method of claim 26, wherein the hyaluronic acid polymers have a molecular weight of from about 0.1 MDa to about 10 MDa.

28. The method of claim 24, wherein the biodegradable polymers are functionalized with cross-linking groups.

29. The method of claim 24, wherein the biodegradable scaffold is porous and has an average pore size of from about 20 micron to about 200 micron.
